(19) United States
(12) Patent Application Publication
(10) Pub. No.: US 2011/0014297 A1
(43) Pub. Date: Jan. 20, 2011

(54) MULTIMODAL THERAPEUTIC HYBRID PARTICLE COMPLEX AND SYSTEM

(75) Inventors: Abraham Phillip Lee, Irvine, CA (US); Kanaka Hettiarachchi, Menlo Park, CA (US)

Correspondence Address: MURABITO, HAO & BARNES, LLP, TWO NORTH MARKET STREET, THIRD FLOOR, SAN JOSE, CA, 95113, US

(73) Assignee: THE REGENTS OF THE UNIVERSITY OF CALIFORNIA, Oakland, CA (US)

(21) Appl. No.: 12/890,243

(22) Filed: Sep. 24, 2010

Related U.S. Application Data
(63) Continuation-in-part of application No. 12/247,782, filed on Oct. 8, 2008.
(60) Provisional application No. 61/245,949, filed on Sep. 25, 2009.
(60) Provisional application No. 61/254,583, filed on Oct. 23, 2009.

(51) Int. Cl. A61K 9/14; A61K 9/00; A61K 38/16; A61K 38/17; A61M 37/00

(52) U.S. Cl. 424/491; 424/400; 514/21.2; 514/20.9; 604/23

(57) ABSTRACT

A multiple layer microbubble drug delivery system, multiple layer microbubble drug delivery vehicle, method thereof and fabrication method are disclosed. The microfluidic drug delivery system for producing multiple layer microbubbles includes a first inlet which receives a gas and directs the gas into a central stream, a second inlet which receives a first liquid containing a drug substance and flow focuses the first liquid around the gas, a third inlet which receives a second liquid and flow focuses the second liquid around the first liquid and a fourth inlet which receives a third liquid and flow focuses the third liquid around the second liquid. The multiple layer microbubble drug delivery vehicle includes a gas core, a first liquid layer containing a drug and surrounding the gas core, a second liquid layer surrounding the first liquid layer to stabilize the first liquid layer and a plurality of particles outside the second liquid layer.

### RELATED U.S. APPLICATION

The present application is a continuation-in-part of U.S. patent application Ser. No. 12/247,782, filed on Oct. 8, 2008, entitled “MULTIPLE-LAYER MICROBUBBLE LIPOSPHERE DRUG DELIVERY VEHICLE AND SYSTEM,” which is incorporated herein by reference. This application claims priority to the co-pending U.S. provisional patent application Ser. No. 61/245,949, Attorney Docket Number 2008-707-1, entitled “MULTIMODAL THERAPEUTIC HYBRID PARTICLE COMPLEX AND SYSTEM,” with filing date Sep. 25, 2009, and the co-pending U.S. provisional patent application Ser. No. 61/254,583, Attorney Docket Number 2009-609-1, entitled “POLYMER-PHOSPHOLIPID SHELLED MICROBUBBLES AND THEIR USES,” with filing date Oct. 23, 2009, both of which are hereby incorporated by reference in their entirety.

### STATEMENT REGARDING FEDERALLY SPONSORED RESEARCH AND DEVELOPMENT

The U.S. Government may have a paid-up license in this invention and the right in limited circumstances to require the patent owner to license others on reasonable terms as provided for by the terms of Grant Nos. NIH 1 R21EB005325-01, R01-EB008733 and R03EB006846 awarded by the National Institute of Health (NIH).

### BACKGROUND

One of the biggest limitations with currently available chemotherapeutics is their systemic toxicity. For example, despite years of research, there are still only a few methods available to deliver anticancer drugs selectively to tumor tissues. Intravenous or oral administration may cause severe toxicity, impeding the therapeutic potential of anticancer drugs. Due to this systemic toxicity, many researchers believe the most important goal of anticancer drug delivery is to maximize therapeutic concentration in tumors while minimizing the exposure of normal tissues. In addition to the challenge of site-specificity, an additional challenge with administration of chemotherapeutics is that sub-therapeutic doses of chemotherapeutic may actually cause tumors to develop drug resistance as a result of biochemical changes.

One of the advances in chemotherapeutics to achieve site-specific delivery has been to encapsulate cytotoxic drugs in liposomes. Liposomes can be designed to contain the drug so as to minimize systemic effects. Researchers have been able to achieve some specificity for where these drug-laden liposomes accumulate—preferentially in tumors. Indeed, several major FDA-approved drugs, such as DOXIL, use liposome encapsulation technology. Despite these advantages and demonstration of promise to date, liposomes have the major disadvantage in that their biodistribution after injection is still relatively nonspecific, and accumulation of liposomes by size selection or molecular targeting is a slow process. Also, the commonly used production technique of mechanical shaking creates a highly polydisperse microbubble population, with varying amounts of oil within many of the microbubble vehicles. Thus, the drug dosages within each vehicle are also not consistent.

### SUMMARY

In one embodiment, a microfluidic drug delivery system for producing multiple layer microbubbles is provided. The microfluidic drug delivery system includes a first inlet which receives a gas and directs the gas into a central stream, a second inlet which receives a first liquid containing a drug substance and flow focuses the first liquid around the gas, a third inlet which receives a second liquid and flow focuses the second liquid around the first liquid and a fourth inlet which receives a third liquid and flow focuses the third liquid around the second liquid.

In another embodiment, a multiple layer microbubble drug delivery vehicle is provided. The multiple layer microbubble drug delivery vehicle includes a gas core, a first liquid layer containing a drug and surrounding the gas core, a second liquid layer surrounding the first liquid layer to stabilize the first liquid layer and a plurality of particles outside the second liquid layer.

In another embodiment, a method for treatment of disease in humans is provided. Multiple layer drug delivery vehicles are introduced into blood and the drug delivery vehicles are concentrated at a target site. Radio frequency energy is applied to the target site to focus the drug delivery vehicles.

These and other objects and advantages of the various embodiments of the present invention will be recognized by those of ordinary skill in the art after reading the following detailed description of the embodiments that are illustrated in the various drawing figures.

### BRIEF DESCRIPTION OF THE DRAWINGS

The present invention is illustrated by way of example, and not by way of limitation, in the figures of the accompanying drawings and in which like reference numerals refer to similar elements.

FIG. 1 is a diagram showing an exemplary microfluidic drug delivery system of one embodiment of the present invention.

FIG. 2 is a diagram showing an expanding nozzle of an exemplary microfluidic drug delivery system of one embodiment of the present invention.

FIG. 3 is a diagram showing filtering channels of an exemplary microfluidic drug delivery system of one embodiment of the present invention.

FIG. 4 is a chart showing the relationship between possible microbubble sizes and production rate with different number of flow-focusing chamber arrays for a constant gas and liquid flow rate and a chart showing improvement in microbubble production for an 8 flow-focusing chamber device.

FIG. 5 is a schematic showing fabrication of an exemplary microfluidic drug delivery system of one embodiment of the present invention.

FIG. 6 is a diagram showing an exemplary three-dimensional microfluidic drug delivery system of one embodiment of the present invention.

FIG. 7 is a schematic showing fabrication of an exemplary three-dimensional microfluidic drug delivery system of one embodiment of the present invention.

FIG. 8 is a diagram showing an exemplary multiple-layer microbubble drug delivery vehicle of one embodiment of the present invention which can be produced by the exemplary drug delivery system of one embodiment of the present invention.

FIG. 9 is a diagram showing different possible radii of microbubbles in microns and corresponding translation in microns.

FIG. 10 is a diagram showing different possible radii of microbubbles in microns and corresponding destruction threshold at different pressures.

FIG. 11 is a diagram showing different possible radii of microbubbles in microns and resonant frequency.

FIG. 12 is a diagram showing different possible radii of microbubbles in microns and expansion coefficient (a measurement of acoustic activity).

FIG. 13 is a diagram showing “nearly monodisperse” microbubble population.

FIG. 14 is a schematic showing a functionalized dual-layer vehicle attached to a cell membrane protein via avidin/biotin interaction.

FIG. 15 is a diagram illustrating the displacement of a microbubble as a function of resting radius and acoustic pressure over the duration of a 20-cycle acoustic pulse at 3 or 5 MHz and 100 kPa.

FIG. 16 is a diagram illustrating a hybrid particle complex in which superparamagnetic iron oxide particles (SPIO) and/or superparamagnetic iron oxide nano-particles (SPION) are attached to the exterior of an AAL of one embodiment of the present invention.

FIG. 17 is a diagram illustrating an alternative configuration of a hybrid particle in which magnetic iron oxide particles are contained within the inner oily layer of one embodiment of the present invention.

FIG. 18 is a diagram illustrating a three dimensional cross-section of a hybrid particle complex that includes a targeting ligand on the exterior thereof of one embodiment of the present invention.

FIG. 19 is a diagram illustrating a top view of a complete microfluidic channel network that is used for hybrid particle complex production of one embodiment of the present invention.

FIG. 20 is a diagram illustrating a magnified view of region of interest with arrows corresponding to flow direction.

FIG. 21 is a diagram illustrating a setup used to obtain images of the Oil Blue N dye liberated from the hybrid complexes in response to applied radiofrequency energy of one embodiment of the present invention.

FIG. 22 is a graph illustrating sample temperature as a function of time in response to application of a 775 Gauss alternating magnetic field.

FIG. 23 is a diagram illustrating a hybrid polymer-lipid functionalized microbubble of one embodiment of the present invention.

FIG. 24 is a diagram illustrating cured spherical PLBs that are densely or loosely packed within porous elastic membrane for biosensing of one embodiment of the present invention.

FIG. 25 is a diagram illustrating a PDMS-Schematic of a PDMS-lipid functionalized drug delivery microbubble of one embodiment of the present invention.

FIG. 26 is a diagram illustrating how cured spherical PDMS microbubbles can be densely or loosely packed on-chip based on microfluidic chip geometry at specific locations for biodetection applications of one embodiment of the present invention.

FIG. 27 is a diagram illustrating the geometry of complete device and zone of interest for particle production of one embodiment of the present invention.

FIG. 28 is a diagram illustrating a setup of a flow-focusing device attached to fluid sources on a microscope imaging system and a setup of three sample curing methods of one embodiment of the present invention.

### DETAILED DESCRIPTION

Reference will now be made in detail to embodiments of the present invention, examples of which are illustrated in the accompanying drawings. While the invention will be described in conjunction with these embodiments, it will be understood that they are not intended to limit the invention to these embodiments. On the contrary, the invention is intended to cover alternatives, modifications and equivalents, which may be included within the spirit and scope of the invention as defined by the appended claims. Furthermore, in the following detailed description of embodiments of the present invention, numerous specific details are set forth in order to provide a thorough understanding of the present invention. However, it will be recognized by one of ordinary skill in the art that the present invention may be practiced without these specific details. In other instances, well-known methods, procedures, components, and circuits have not been described in detail as not to unnecessarily obscure aspects of the embodiments of the present invention.

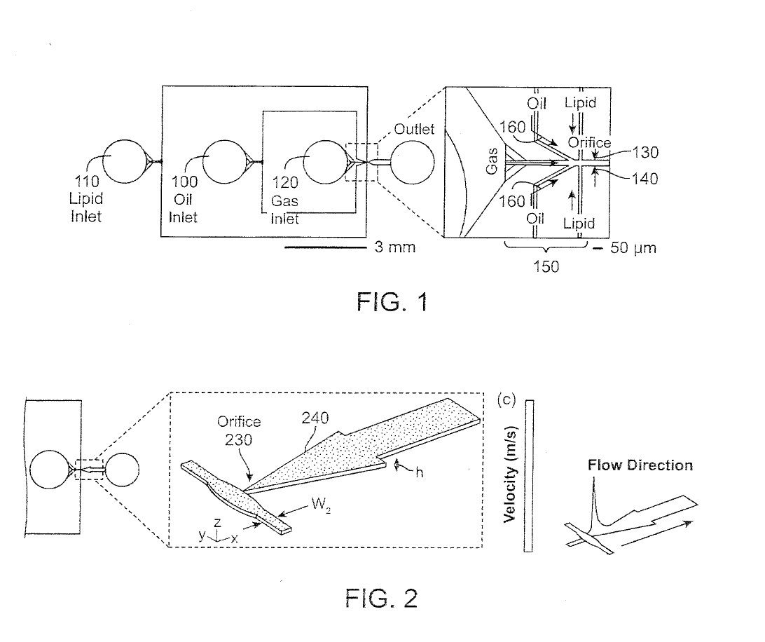

FIG. 1 is a diagram showing an exemplary microfluidic drug delivery system of one embodiment of the present invention. Using the drug delivery system as shown, controlled generation of multiple-layer microbubbles for drug delivery can be generated in a controllable manner. The microbubbles can be liposomes or artificial lipid vesicles which can be loaded with a variety of water-soluble drugs within their inner aqueous compartment or with water insoluble drugs within the hydrophobic interior of the phospholipid bilayer. The liposomes can be readily composed of biologically inert materials and can be easily made biocompatible. The microbubbles can be monodisperse double emulsions and can be a contrast agent. The drug delivery system comprises an inlet 100 for a substance capable of carrying a bioactive substance which can be an oil such as triacetin or a hydrophobic substance to solubolize a hydrophobic drug such as Paclitaxel. In addition to triacetin, a variety of oils such as soybean and safflower oil can be used to dissolve hydrophobic drugs and hydrophobic cancer drugs. Other oils such as mineral oils, vegetable oils, animal oils, essential oils, synthetic oils or other mixtures can be used. An oil rich in triglycerides can be ideal for use with cancer drugs such as Paclitaxel.

The drug delivery system can also comprise a second inlet 110 for a second substance which can be a lipid. The drug delivery system can also comprise a third inlet 120 for a third substance which can be a osmotic agent with significantly different density and compressibility than blood e.g. a gas such as nitrogen. Perfluorocarbon or PFC gas can also be used resulting in polydispersity index (σ) values of <2%. Octafluorocyclobutane gas can also be used. Gas composition can be a factor in determining the length of time a microbubble lasts in the circulation. High molecular weight (>150 Da) gases with low water solubility (<0.2 mol m-3 at 25° C.) such as PFC gasses, perfluoropropane (C₃F₈), perfluorocyclobutane (C₄F₈), perfluorobutane (C₄F₁₀) or perfluoropentance (C₅F₁₂) can be used. The use of high molecular weight gases such as PFCs can reduce diffusion out of the microbubble core and can enhance bubble stability and circulation lifetime by counterbalancing the Laplace and blood pressures.

Standard liposomes are not acoustically active (or at most, very weakly acoustically active) because their density and compressibility are similar to the surrounding blood. Acoustically active drug carriers should possess the combination of a layer with drug-carrying capacity, yet at the same time, they should have a core with significantly different density and compressibility than the surrounding blood—such as a gas. There are two primary ways to design these combination vehicles —either to surround the bubble with a thick layer which can incorporate the drug, or to attach drug carriers such as liposomes directly to the outside of the bubbles. A vehicle with the drug carrying capacity of a liposome, but the acoustic activity of a microbubble can be readily steered and concentrated at the target site by ultrasound, reducing the need to rely on molecular targeting or passive mechanisms. Disruption of the acoustically-active vehicles at the target site can result in sub-micron fragments which can then extravasate in leaky tumor microvasculature. Improved chemotherapeutic delivery, with reduced systemic toxicity, can be achieved by utilizing ultrasound to concentrate and disrupt acoustically-active drug delivery vehicles at a target site. Additionally, acoustically active drug delivery vehicles can be directly imaged by ultrasound, providing instant feedback as to their location.

Using the drug delivery system of one embodiment of the present invention, multi-layer acoustically-active drug delivery vehicles can be precision engineered. Additionally, vehicles with a tightly controlled size distribution, drug content, and consistent stability can be produced. The application of microfluidics can be used to produce precision-tuned contrast agents with a monodispersity far better than has been demonstrated by traditional bulk-manufacturing methods to date. Precision engineered acoustically active drug delivery vehicles can possess uniform acoustic characteristics, and uniform drug payload. Vehicles which possess identical acoustic characteristics can be easier to concentrate and disrupt with ultrasound, compared to polydisperse vehicles. The ability to accurately control the amount of drug in each vehicle has not been possible in the past. The drug delivery system of one embodiment of the present invention can improve the quality control of drug delivery vehicles. The appropriate microfluidic channel geometries, device materials (e.g. PDMS), and surface coating methods can be chosen so parameters such as vehicle diameter, drug and gas content, and shell structure can be tightly controlled.

As shown, the drug delivery system can comprise combined dual flow-focusing region for one or more of the inlets and expanding nozzle geometry 140 for one or more of the inlets. The flow focusing region 150 can be hydrodynamic. An orifice 130 with a width of 15 μm can be utilized. The drug delivery system can produce microbubbles by flow-focusing, forcing a central stream of gas and two (lipid and oil) double-sided liquid sheath flows through a narrow orifice 130. The drug delivery system can produce liposphere microbubbles of 10 μm which can be an ideal size for carrying large drug payloads. It can also produce liposphere microbubbles of 10 μm or less which is a suitable size range for oral drug delivery or direct injection into the circulation. For therapeutic use, microbubbles of sizes between 50 to 1000 nanometers can be used. The gas can be supplied from a pressurized tank via flexible tubing and delivered into the chamber using a micro flow meter. The continuous lipid and oil phase mixtures can be pumped at constant flow rates using two digitally controlled syringe pumps. A Nikon inverted microscope and high-speed camera can be used to capture images and record movies of the dual-layer microbubble lipospheres in the microfluidic system.

The channels for lipid, oil and gas can have a rectangular cross section and a height of 25 μm. The widths of the lipid, oil, and gas inlet channels can be 50 μam, 15 μm and 35 μm, respectively. The drug delivery system can have an expanding nozzle 140 with an orifice width of 15 μm or 7 μm. The outlet channel can connect to an open reservoir for collection. The arrows indicate direction of flow.

FIG. 2 is a diagram showing an expanding nozzle of an exemplary microfluidic drug delivery system of one embodiment of the present invention. All channels have a rectangular cross section. Three dimensional rendering of the flow-focusing expansion chamber is shown. The devices feature an expanding nozzle 240 with a range of orifice widths. A three dimensional surface plot with height showing the velocity field in the plane of the microfluidic device is also shown. The drug delivery systems forces a central stream of a dispersed phase and two side sheath flows of a continuous phase mixture through a narrow orifice 230 into a second chamber. The focusing effect of the surrounding flow of liquid breaks the thread at the orifice 230 into uniform emulsions. Expanding nozzle 240 geometry generates monodisperse emulsions, focusing the break-off location to one single point located at the orifice 230. The narrowest point incurs the highest shear force, and the subsequent nozzle expansion generates a velocity gradient in the flow direction that allows the head of the thread to break continuously at the orifice 230, which provides uniform control of emulsion sizes.

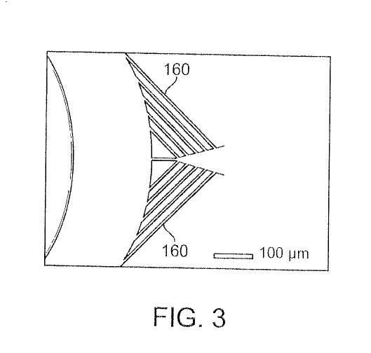

FIG. 3 is a diagram showing filtering channels of an exemplary microfluidic drug delivery system of one embodiment of the present invention. All channels have a rectangular cross section and a height of 25 μm. The widths of the liquid and gas inlet channels are 50, 75, 100 μm and 35, 50 μm respectively. The devices can have an expanding nozzle with a range of orifice widths (7, 10 15, 20, 25 μm). The outlet channel connects to an open reservoir for bubble collection. Main functional area can have a 7 μm orifice and 3 μm microbubble generation. Magnified diagram of liquid inlet (left) and gas inlet (right) filtering channels 160 is shown.

A single flow-focusing microfluidic drug delivery system can be limited to a production rate only on the order of 10¹⁰ per minute. To increase microbubble production while maintaining size-stability, microfluidic devices containing two, four, and eight chamber flow-focusing arrays can be utilized. PDMS-based microfluidic manufacturing allows the manufacturer to inexpensively integrate and multiplex several flow-focusing circuits together, resulting in higher throughput and speed in microbubble production. With this planar channel network configuration, parallel arrays of bubble generation chambers less than a millimeter apart is possible.

In the exemplary arrayed microfluidic drug delivery system of one embodiment of the present invention, an array of eight single-orifice flow-focusing chambers are placed in parallel to another array between a central gas inlet. The path lengths from each of the eight flow-focusing chambers to the gas and liquid phase inlets are the same. This helps ensure monodisperse bubble production in all eight chambers based on geometry. Incorporating only two controllable inputs (gas and liquid) instead of the normal sixteen for eight independent flow-focusing chambers greatly reduces the complexity of the system, eliminating potential errors due to fluctuations from multiple sources.

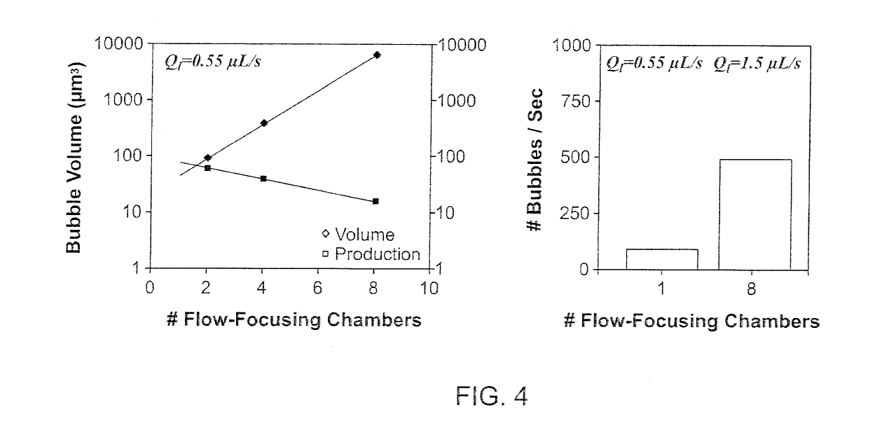

FIG. 4 is a chart showing the relationship between possible microbubble sizes and production rate with different number of flow-focusing chamber arrays for a constant gas and liquid flow rate and a chart showing improvement in microbubble production for an eight (8) flow-focusing chamber device.

The generated size of microbubbles increases exponentially and production rate decreases as the array number doubles when using the same liquid flow rate and gas pressure. Higher liquid flow rates are therefore required when using multi-arrays to increase production of lipid-stabilized microbubbles and maintain the same microbubble size as in a single chamber system. A nearly six-fold increase in the volumetric rate capacity over a single chamber system can be achieved from a system with eight flow-focusing chambers. The discrepancy is largely due to the gas permeability of PDMS, an elastomeric polymer with flexible Si—O linkages. This leads to high diffusion coefficients as compared to C—C backbone of many organic polymers. For larger arrays, applying higher pressures can result in more gas penetration through the entire polymer matrix rather than being focused through the channel networks. An organic polymer or more robust material such as polyurethane or PU, an elastomeric polymer with some glassy characteristics, can further improve production by reducing gas permeability.

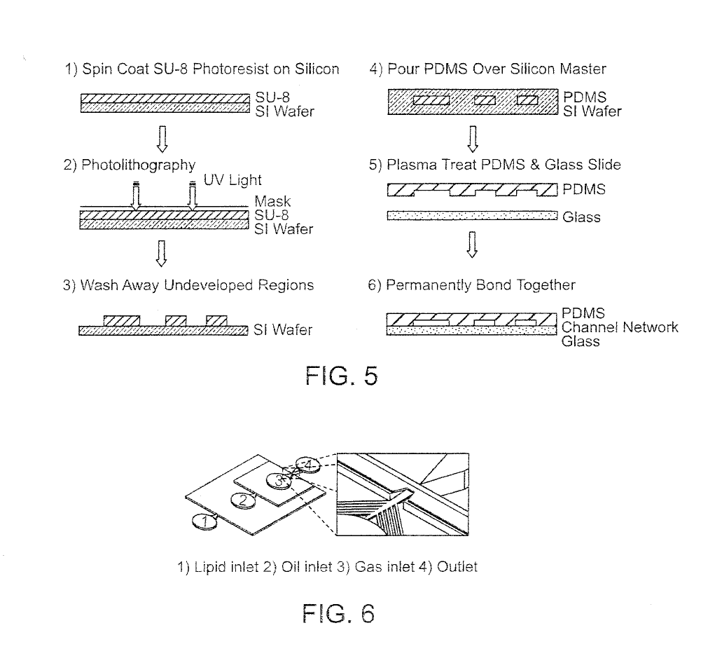

FIG. 5 is a schematic showing fabrication of an exemplary microfluidic drug delivery system of one embodiment of the present invention. The drug delivery system can be fabricated from a SU-8 master with the channels molded in poly(dimethylsiloxane) (PDMS) using soft lithography and rapid prototyping techniques. A high resolution 20,000 dpi photomask enables designs having critical channel widths such as a 15 μm orifice and closely spaced filtering channels.

Soft lithography can be accomplished using a set of methods for fabricating structures using elastomeric stamps. An optically clear, inert, gas permeable, and non-toxic material such as the elastomeric material poly(dimethylsiloxane) or PDMS can be used for flow delivery. The method is well-suited for biomedical applications, and advantages included the ability to construct small micron-scale features and lower costs with mass production. A silicon wafer is spin-coated with a layer of a UV-curable epoxy and exposed to UV-light through a high resolution photomask containing the channel pattern and developed. The wafer is used to cast a replica in PDMS consisting of a 10:1 pre-polymer and curing agent ratio and bonded to clean soda lime glass (Corning) after oxygen plasma treatment. The surface hydrophobicity can be adjusted depending on the application. In certain conditions when the flow pressure is high, PDMS may not be the optimal material of choice.

The lipid shell can be a phospholipid such as 1,2-distearoyl-sn-glycero-3-phosphocholine or DSPC. The lipid shell can also be a lipopolymer emulsifier such as 1,2-distearoyl-sn-glycero-3-phoshoethanolamine-N-[Poly(ethylene glycol)2000] or DSPE-PEG2000 or a combination of DSPC and DSPE-PEG2000. A recipe for lipid preparation consisting of adding to a vial containing a 9:1 molar ratio of phospholipid to emulsifier, a 10% aqueous glycerol/propylene glycol mixture, can be used. The lipids DSPC and DSPE-PEG2000 can be combined at 9:1 molar ratio, dissolved in chloroform and exposed to nitrogen and vacuum to create a homogenous mixture. The aqueous glycerol/propylene glycol mixture with the stabilizing lipids DSPC and DSPE-PEG2000 can be used for the outer sheath flow stream. An oil soluble blue dye at 1 mg/mL for microscopy studies can be added. The fluorescent probe DiI-C18 (1,1′-dilinoleyl-3,3,3′,3′-tetramethylindocarbocyanine perchlorate, Molecular Probes) can be added at 1 mol % for fluorescence microscopy studies. Water, purified using a Millipore system can added to the vial containing the lipid mixture, sonicated at room temperature for 20 minutes, and combined with a 10% aqueous glycerol/propylene glycol (GPW) mixture.

Mixed monolayers of DSPC (1,2-distearoyl-sn-glycero-3-phosphocholine) and DSPE-PEG2000 (1,2-distearoyl-sn-glycero-3-phoshoethanolamine-N-[Poly(ethylene glycol)2000]) at 89/10 ratio of mole percentages or DPPC (1,2-dipalmitoyl-sn-glycero-3-phosphocholine), DPPA (1,2-dipalmitoyl-sn-glycero-3-phosphate) and DPPE-PEG5000 (1,2-dipalmitoyl-sn-glycero-3-phosphoethanolamine-N-[methoxy(polyethylene glycol)-5000]) at 81/8/10 ratio of mole percentages as the primary shell components, doped with the fluorescent probe DiI-C18 (1,1′-dilinoleyl-3,3,3′,3′-tetramethylindocarbocyanine perchlorate) at 1 mol % can also be used. These blends of lipid can optimally stabilize the microbubbles and can confer a slight negative charge to the membrane, reducing interaction between adjacent vehicles.

The oil layer can be a drug or therapeutics carrier such as Glyceryl triacetate or Triacetin used in the inner sheath flow stream. The drug used can be a therapeutic compound, cytotoxic drug, chemotherapy agent, chemotherapeutic drug, DOXIL or Paclitaxel. DOXIL is a long-circulating PEGylated liposome containing the chemotherapeutic doxorubicin, which can be used to treat metastatic breast carcinoma, unresectable hepatocellular carcinoma, cutaneous T-cell lymphoma, sarcoma, squamous cell cancer of the head and neck, and ovarian cancer. Paclitaxel binds to the β subunit of tubulin, preventing depolymerization of microtubules. Paclitaxel can be dissolved at high concentrations, such as 70 mg/ml, in the lipid-oil complex of the liposphere microbubble. The liposphere microbubble vehicles can provide an alternative method of delivery of therapeutic drugs that have poor oral bioavailability. Paclitaxel is a poorly water soluble drug and its large size prevents it from being accommodated within the phospholipid bilayers in liposomes. In a dual-layer vehicle, a high volume fraction of the oil phase can be used and paclitaxel exhibits good solubility in triacetin. Delivery of more precise dosages of drug can be enabled.

Although molecular targeting is not required for acoustically-targeted delivery, the surfaces of acoustically active drug delivery vehicles can be functionalized. Microbubble vehicles can be further developed by having specific targeting ligands, biotin, biotin groups, -amine groups or specific peptide such as RGD on the shell surface for targeting applications or attachment of bioconjugates. Conjugating a cyclic-RGD peptide to the shell due to over-expression of αvβ3 integrin in the tumor blood vessel endothelium can be used for targeted delivery. Vehicles bearing a phosphatidylserine or PS or having the lipid component 1,2-distearoyl-sn-glycero-3-phosphochoserine or DSPS may be used to target or treat plaques. Targeting may also include vehicles functionalized to bind to p-selectin and αvβ3 integrin on the cell membrane. Such vehicles can favorably target tumors due to over expression of cell surface receptors. Targeting capability can be beneficial for vehicles that carry chemotherapeutic drugs.

Since most chemotherapeutic agents have inherently severe systemic toxicity, it can be desirable to minimize non-specific drug-carrier accumulation. One mechanism that can be used for drug carrier localization is ultrasound. Ultrasound can concentrate particles and their contents through two mechanisms of action directly on the particles—radiation force, and particle disruption.

Acoustic radiation force, produced by an ultrasound wave traveling through a lossy medium, causes particles within the medium to travel in the direction of the sound wave. This force can have the effect of causing physical localization of particles as determined by the ultrasound direction and focus. While incompressible objects do experience radiation forces, compressible objects driven at their resonant frequency experience larger forces and can be observably displaced by low-amplitude ultrasound waves. Thus, vehicles can be designed to have a large compressibility and a resonance frequency in the frequency range of clinical ultrasound systems. Small microbubbles—also used as ultrasound contrast agents—can be readily made with characteristics that are optimized to experience radiation force. Ultrasound radiation force can be used as a mechanism for site-specific particle localization—without relying on inherent properties of the target tissue such as permeability and expression of adhesion ligands.

Acoustic radiation force can be significant on microbubbles at clinical imaging frequencies. This force can be maximized by tuning the center frequency of the ultrasound to the resonant frequency of the contrast agent microbubbles. Improving the monodispersity of the microbubble or drug delivery vehicle with microfluidics can vastly increase the percentage of the population that can be effectively manipulated by radiation force. A bubble of 1.7 μm in diameter, which has a resonant frequency at 5 MHz, can be displaced by radiation force five times further than a bubble of 3 μm in diameter, which has a resonant frequency at 2.5 MHz, in response to a 5 MHz acoustic pulse. A streamline of contrast agents can be moved over 100 μm at a low acoustic pressure, and contrast agents can be displaced over a centimeter with optimized parameters.

Radiation force can localize contrast agents along the wall of small arterioles and venules, tissue, and in the chorioallantoic membrane. Contrast agents circulating in the blood pool can be brought into contact with the endothelium. Additionally, when agents are concentrated near a vessel wall, they can travel at a reduced velocity compared to those in the center of the flow stream. In cooperation with primary radiation force, which acts in the direction of acoustic wave propagation, a secondary radiation force for which each individual bubble is a source and receptor can cause the agents to attract each other. The result of this secondary force is that a much larger concentration of microbubbles collects along a vessel wall than might otherwise occur. The effects of radiation force are desirable for maximizing efficiency of adhesion receptors on targeted agents. Radiation force can increase the adhesion of targeted ultrasound contrast agents over 20 fold and radiation force can assist the localization and adhesion of molecularly-targeted microbubbles.

After localization of acoustically-active drug delivery vehicles by molecular targeting or radiation force, ultrasound can be utilized to cause fragmentation of the carrier vehicles into sub-micron droplets for delivery. Triacetin oil nanodroplets created after fragmentation of acoustically-active lipospheres can be an effective method of transferring paclitaxel, a chemotherapeutic or a fluorophore to target cells. The ability to cause this disruption is related to both the acoustic parameters, the vehicle size, and other properties of the vehicle such as viscosity. Using ultrasound, the drug carrying layer can be fragmented into nanodroplets. A sub-micron droplet size of the nanodroplets can be used for extravasation through tumor microvasculature.

FIG. 6 is a diagram showing an exemplary three-dimensional microfluidic drug delivery system of one embodiment of the present invention. Two-dimensional systems can face challenges for producing consistently sized dual-layer microbubbles due to the large viscosity difference between the oil and gas. The formation of oil droplets along with the generation of microbubble lipospheres have been observed in two-dimensional systems. This indicates that the gas phase is not entirely surrounded by the oil phase, leading to oil droplets being formed along with lipid microbubbles. A three-dimensional flow-focusing system as shown where the gas phase is surrounded entirely by the continuous oil and lipid phase can result in more stable thread break-up and multi-layer vehicle formation, resulting in better size consistency.

Three-dimensional hydrodynamic focusing can enable more precise positioning of molecules in both vertical and lateral dimensions, constraining the sample flow to the center of the channel, minimizing the interaction of the sample with the surfaces of the channel walls. A three-dimensional design can enable smaller diameters and precision size tolerance for the formed bubbles. Having the gas phase surrounded entirely by the continuous oil and lipid phase can also result in more stable thread break-up and multi-layer bubble formation, and can result in better size consistency. Three-dimensional flow focusing can minimize surface effects, thus enabling longer and more stable run times.

A three-dimensional microfluidic flow-focusing system can be fabricated using a PDMS sandwich. PDMS is an optically clear, flexible material that can be stacked onto other cured polymer pieces to form advanced microstructures with complex geometries. In order to create the master mold for the PDMS containing channels of varying height, a high energy beam sensitive glass (HEBS-glass) as the gray level mask can be used. Using such a gray level mask, alignment errors can be avoided following optical exposure since the mask is written in a single step using different electron beam dosages to generate gray levels. This can eliminate the need for three binary masks of three different channel heights (lipid, oil, and gas) and also can avoid repeated resist processing and wet etching.

Multiplexing can enhance the capability of microfluidic systems that generate microbubbles with several times the volumetric rate capacity of single systems. The ability to multiplex can be important for scale-up production of contrast agents used in large-scale environment. Two, four, and eight chamber flow-focusing arrays in devices with three-dimensional channel systems of varying heights can be used. The single-orifice flow-focusing chambers can be multiplexed in the radial direction away from the gas input. The three-dimensional channel network can be fabricated on a 3″ silicon wafer using standard soft lithography and rapid prototyping methods with a HEBS gray level mask.

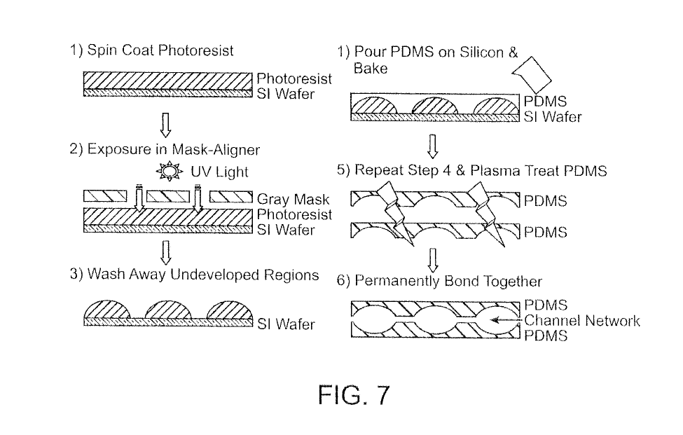

FIG. 7 is a schematic showing fabrication of an exemplary three-dimensional microfluidic drug delivery system of one embodiment of the present invention. Soft lithography and rapid prototyping steps in microfluidics using a gray scale mask is shown, providing for convenient formation and manufacturing of three-dimensional microstructures. An elastomeric stamp (PDMS) contains the patterns and structures with feature sizes ranging from 5 μm-25 μm. A HEBS-glass mask having a minimum of 96 gray levels can be used to fabricate channel heights of 5 μm, 10 μm, and 25 μm respectively. Thick photoresist (Shipley S1650) can be spin-coated on two, 3-inch silicon wafers to achieve a resist thickness in the range 25 microns. Upon contact alignment, each wafer with photoresist can be exposed to UV-light through a gray-scale HEBS mask containing the channel pattern and developed. The wafers can be used to cast a replica in poly(dimethylsiloxane) PDMS consisting of a 10:1 prepolymer and curing agent ratio. After overnight baking in a 65° C. oven, the PDMS can be bonded together after a 2 minute oxygen plasma treatment. The surface hydrophobicity can be adjusted depending on the application. The exemplary three-dimensional microfluidic drug delivery system of one embodiment of the present invention can minimize surface effects, which can compromise the generation of monodisperse bubbles in two-dimensional systems. This can enable longer and more stable run times, and also precise positioning and better characterization of the acoustically active delivery vehicles.

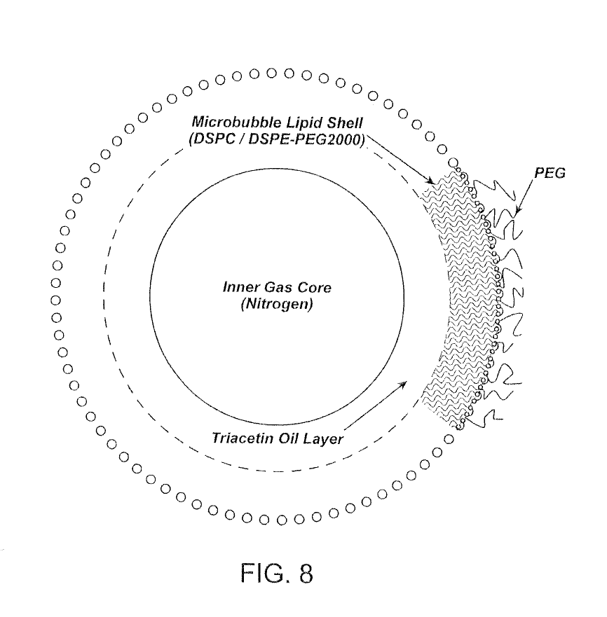

FIG. 8 is a diagram showing an exemplary multiple-layer microbubble drug delivery vehicle of one embodiment of the present invention which can be produced by the exemplary drug delivery system of one embodiment of the present invention. The microbubbles can be lipospheres and can be gas-filled vehicles with a lipid shell and an additional layer of oil just beneath the lipid shell. The thickness of lipid/oil layers are not to scale with diameter of the overall vehicle. The gas allows for the microbubbles to be acoustically active and the lipid-oil complex allows delivery of bioactive substances at high concentrations. The oil layer can be capable of carrying highly hydrophobic drugs such as the chemotherapeutic drug Paclitaxel, which is currently delivered to cancer patients intravenously.

The outer DSPC lipid shell stabilizes the vehicle and the inner oil layer can contain dissolved therapeutics. The lipid shell can incorporate PEG to reduce immunogenicity. The gas interior makes the vehicle acoustically active to ultrasound and ultrasound pulses. Once attached to cells, the applied radiation force will cause vehicle disruption and drug release.

Microbubble size and production rate are highly prone to downstream pressure conditions. The large viscosity difference between the oil and gas should be accounted for in the production of dual-layer liposphere microbubbles. Channel geometry in addition to the liquid and gas flow rates are the main factors used to control the liposphere microbubble sizes. The viscosity of triacetin (28.0cP) is considerably less compared to other oils, making it desirable for use at liquid flow rate regimes of 0.5-1.0 μL/second. Flow rates of 0.5, 1.0, 1.5 and 2.0 μL/second can also be achieved.

There was no observed change in the size of lipid coated microbubbles from minutes to hours after generation. Although highly size stable for more than two weeks, the polydispersity of lipid coated microbubbles can be >50% when using high liquid flow rates and gas pressures (liquid flow rate Q>1.0 μL/s, gas pressure P>10 psi) to increase production. Increasing Q and P decreases the distance between exiting bubbles, and these contact interactions can cause them to coalesce due to the lower shell resistance in a high flow velocity environment as in the expansion chamber. In addition, DPPC and DSPC lipids exist as liposomal particles in the aqueous continuous phase, and their opening up and spreading as a monolayer at the gas/liquid interface upstream of the orifice, a dynamic adsorption process, can be affected at high rates of flow. Using lower Q and P (Q<1.0 μL/s, P<5 psi) can result in highly monodisperse and stable 5 μm lipid coated microbubbles.

Based prior studies of the effect of microbubble diameter, the size distribution of the vehicle can be extremely important. Parameters such as the destruction threshold of a vehicle, the amount of radiation force experienced, the resonant frequency of the vehicle (important for imaging), and the biodistribution can be significantly affected by the vehicle diameter. Vehicles can be in the 1-5 micron range, which can be an ideal range for contrast agents and delivery vehicles, but where all of these parameters can change drastically with small changes in diameter. Larger bubbles can be produced as well, although they may not be desirable for imaging.

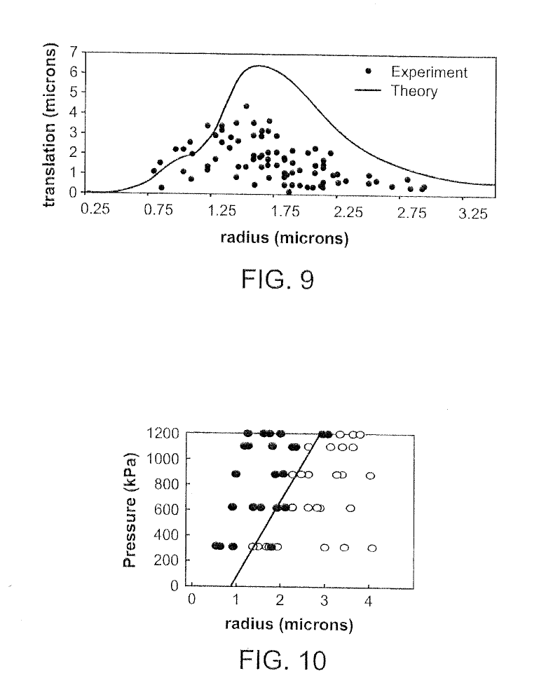

FIG. 9 is a diagram showing different possible radii of microbubbles in microns and corresponding translation in microns.

FIG. 10 is a diagram showing different possible radii of microbubbles in microns and corresponding destruction threshold at different pressures.

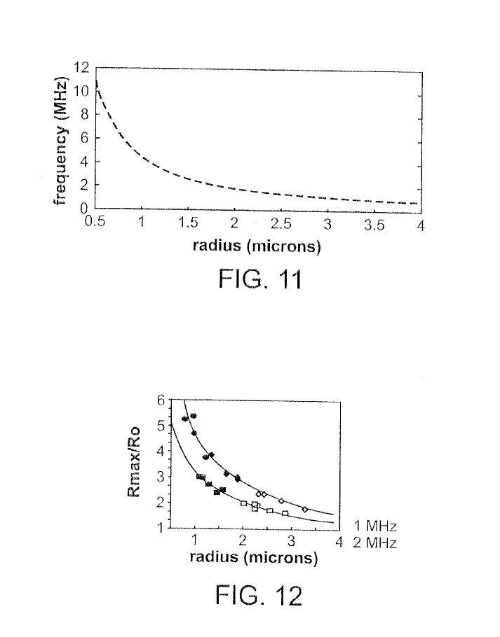

FIG. 11 is a diagram showing different possible radii of microbubbles in microns and resonant frequency.

FIG. 12 is a diagram showing different possible radii of microbubbles in microns and expansion coefficient (a measurement of acoustic activity).

Prior contrast agents can have a polydisperse size distribution such as 2.1±1.1 microns. A microbubble contrast agent with such a distribution was found via simulation to have only 49% of microbubbles whose resonant frequencies lie within the −6 dB bandwidth of a transducer with an optimized center frequency of 3.8 MHz. This result indicates the “best case” for the transducer bandwidth overlapping the resonant frequency distribution of the microbubble population. For higher imaging frequencies such as 5, 7, and 10 MHz, which are more realistic in current contrast imaging, the percent of microbubbles with resonant frequencies within the bandwidth can decrease to 36%, 23%, and 18%, respectively. Using the drug delivery system of one embodiment of the present invention, monodisperse microbubbles of any diameter desired within 1 and 50 microns, with a standard deviation of only 5% of the mean diameter can be produced. This result means that 100% of microbubbles can have their resonant frequency within the transducer bandwidth, and all of the microbubbles can respond to radiation force and disruptive pulses in the same manner. This can result in a several-fold increase in the amount of the population of vehicles that are affected by ultrasound—to nearly 100%. Thus, a precision engineered vehicle can be much more consistent in its radiation force, drug release characteristics, and imaging characteristics than a polydisperse vehicle. Precision engineering with microfluidics can precisely tailor the quantity of drug incorporated into each vehicle, and can ensure that the quantity is precise across the population.

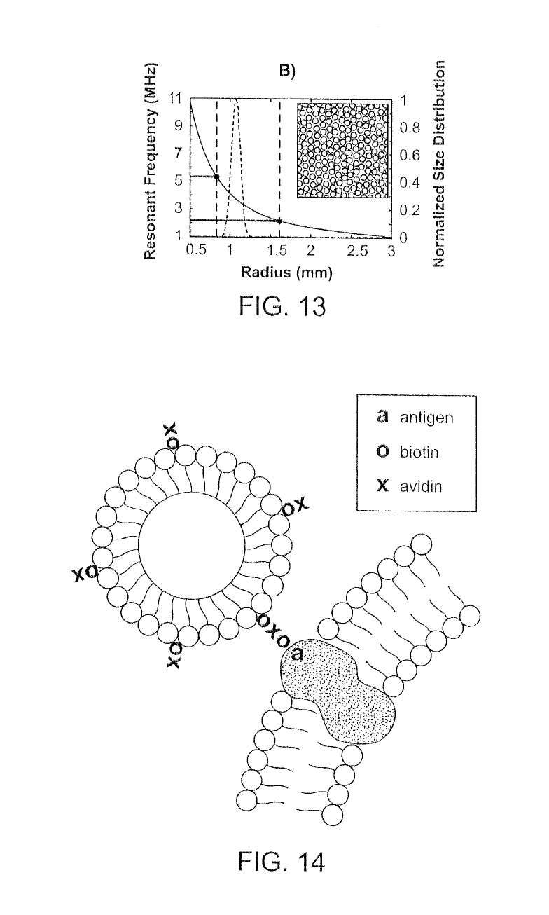

FIG. 13 is a diagram showing “nearly monodisperse” microbubble population. As shown, the mean diameter has a resonant frequency which matches the transducer center frequency, with a standard deviation of 5% of the mean.

An additional major advantage of using microfluidics of one embodiment of the present invention is that the potential also exists for manipulating the chemistry and material characteristics of droplets or particles within microfluidic devices so that different functional properties are added for a given application. The generation of controlled populations of encapsulated microbubbles for ultrasound contrast agents, functionalization of these agents for molecular targeting, and production of multi-layer drug carriers can be achieved. The generation of uniform emulsions in flow-focusing microfluidic devices can provide a new means for the synthesis of novel materials ranging from drugs and vesicles with tailored properties from micro- to nano-particles, and even the controlled construction of porous structures. These applications of microfluidics can have important implications for localized drug delivery applications.

FIG. 14 is a schematic showing a functionalized dual-layer vehicle attached to a cell membrane protein via avidin/biotin interaction. Biotin groups on the lipid shell can be introduced for attachment to cancer cells such as breast cancer cells. Biotinylated targeting ligands can be conjugated to contrast agents functionalized with biotin through an avidin linker. The avidin/biotin interaction is a strong noncovalent binding between protein and ligind. Multi-layer liposphere microbubbles can be functionalized incorporating biotinlyated lipid into the bubble shell and attaching the agent via an avidin linker. Lipid solutions can be prepared as described above, with addition of DSPE-PEG2000-Biotin or 1,2-distearoyl-sn-glycero-3-phoshoethanolamine-N-[Biotin(Polyethylene glycol)2000], Avanti Polar Lipids. The lipids solutions can be composed of 0.5 mg/ml DSPC with 90/5/5 mol/mol/mol ratio of DSPC/DSPE-PEG2000/DSPE-PEG2000-Biotin. 10 μl of Avidin or ImmunoPure Avidin, Pierce can be added to 500 μl of biotinylated microbubbles. After waiting for 5 minutes, the microbubble solution can be washed with deionized or DI water for several times. Biotin or EZ-Link Sulfo-NHS-LC-Biotin, Pierce binds with high affinity to avidin and can be used for labeling of cell surface proteins.

Improvements in the acoustic response of precision engineered microbubbles can be achieved due to their uniform size distribution. Echoes from single microbubbles produced using microfluidics (nearly monodisperse) or by the traditional agitation method (Definity-polydisperse) can be acquired using a custom ultrasound system. Echo to echo correlation, indicates that acoustic responses from precision engineered microbubbles are significantly more correlated than traditional agents. Analysis of m-mode data indicates that the monodisperse agent presents approximately twice the SNR of the polydisperse agent.

A Tektronix AWG2021 arbitrary waveform generator can be used to produce a driving pulse which could be controlled for amplitude, length, shape, and frequency. This driving pulse can be amplified with an ENI RF amplifier, and used to excite an ultrasonic transducer. Microbubbles can be pumped through a 200 micron cellulose tube with a syringe pump (Harvard Apparatus, Hollister, Mass.) at a mean velocity of 20 mm/s. The concentration can be reduced by dilution until single microbubble echoes are observed. The tube can be held vertically to reduce microbubble accumulation along the wall due to floatation and the transducer can be placed at an angle of about 60 degrees with respect to the tube to eliminate reflected echoes from the tube wall. Received echoes can be amplified by 40 dB, bandpass filtered between 1 and 12 MHz, digitized at 125 MHz on a Signatec PDA14 A-D board through a LabView (National Instruments Corporation, Austin, Tex.) interface. The echoes can be analyzed offline with MATLAB. The RMS (root mean square) value of each echo can be calculated directly from the echo in time domain. Additionally, the correlation coefficients between each echo can be calculated and averaged.

Fifty-five echoes from monodisperse and 109 echoes from polydisperse microbubbles were captured and compared. The standard deviation of the RMS echo amplitude for the polydisperse contrast agent was substantially greater than that for the monodisperse agent. The correlation of the echoes from the monodisperse microbubble population was significantly greater than that from polydisperse microbubble population (0.97 vs. 0.70, p<0.0001).

For small, highly compressible objects, such as microbubble contrast agents, the radiation force, produced by insonation with clinical imaging parameters, can result in rapid translation of contrast agents. In addition, secondary radiation force can produce an attractive force between the agents, causing the bubbles to aggregate. The effect of radiation force on a contrast agent is maximized at the agent's resonant frequency.

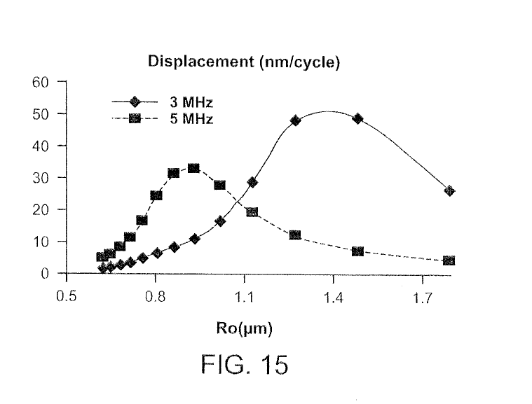

FIG. 15 is a diagram illustrating the displacement of a microbubble as a function of resting radius and acoustic pressure over the duration of a 20-cycle acoustic pulse at 3 or 5 MHz and 100 kPa. By using a monodisperse contrast agent in conjunction with insonation at the resonant frequency, more effective displacement and concentration can be achieved than with a contrast agent with a wide size distribution, where most of the microbubbles will be excited off-resonance.

The acoustic field can cause contrast agents to localize along the wall of a 50-μm arteriole in a cremaster muscle. 50-micron arteriole can be injected with fluorescently-labeled contrast agent and insonation at 5 MHz, 800 kPa, at 10 kHz pulse repetition frequency. These acoustic parameters can generate radiation force sufficient to cause the localization and concentration of contrast agents along the vessel wall opposite from the ultrasound source. Enhancement of adhesion of targeted microbubbles (TC) in a synthetic vessel can be achieved with radiation force (RF). When contrast agents are concentrated near the vessel wall, they travel at a reduced velocity compared to those in the center of the flow stream. Contrast agents shown along the wall of the vessel are traveling less than 1 mm/second, whereas the velocity in the center of the vessel is estimated at 7.5 mm/second. Radiation force can also cause the contrast agents to attract each other, resulting in a much larger concentration of microbubbles along the vessel wall than might normally occur. This combination of localization and concentration of agents near a vessel wall and reduced flow velocity can assist the adhesion of targeted microbubbles. The application of radiation force and targeted contrast agents can be combined for radiation force to assist targeting. The adhesion of biotin-targeted microbubbles to an avidin-coated 200-μm vessel can be increased by over 20 fold by the application of radiation force. The application of radiation force with non-targeted agents do not demonstrate binding, indicating that radiation does not increase non-specific adhesion.

A microinjector to pump individual contrast agent microbubbles into an acoustically and optically transparent 200 micron cellulose tube can be used to determine the acoustic parameters required to disrupt microbubbles. An ultrasonic transducer and microscope objective can be mutually focused on the tube sample volume. The ultrasonic transducer can be excited with an acoustic pulse with an arbitrary waveform generator and a RF amplifier. The arbitrary waveform generator allows testing of pulses of different frequency, amplitude, pulse length, and phase. Optical microscopy with a high-resolution video system allows documentation of the fragmentation or lack thereof of the test agent as a function of acoustic parameters.

In order to determine whether or not drug composition remains with test drug delivery vehicles over time, formulations can be separated using two fractionalization techniques. A fluorescent drug composition, such as Oregon Green Paclitaxel can be used for measurement. Amicon Bioseparations YM-30 30,000 MWCO centrifuge filters can be used as first separation technique. 50 μl of the vehicle formation can be mixed with 250 μl of plasma or PBS. The mixture can be incubated for a set time and temperature, and then in a swinging bucket centrifuge for 15 minutes at 4400 RPM. Samples can be tested at 20° C. and 37° C. for 5, 75 and 180 minutes. The second separation technique can involve fractionalization with a 10 cm long, 1 cm bore column loaded with Sephadex G75. 150 μl of the droplet formation can be mixed with 150 μl of PBS and eluted through the column. Eluted samples can be accumulated every 250 μl.

DiD and DiR samples can be imaged with a Tecan Safire2 plate reader and Oregon Green samples can be imaged with a Biotek FLx 800 plate reader. For DiD readings, excitation and emission can be set to 644 and 655 nm, respectively. For DiR, excitation and emission can be set to 750 and 800 nm, respectively. Oregon Green Paclitaxel can be imaged using 495 and 520 nm for excitation and emission, respectively. The elution rate of the vehicles and free drug can occur at different rates. Small molecules such as the free drug/dye can elute more slowly. Collected aliquots analyzed by the plate reader can provide information as to whether or not the fluorophore remains with the vehicle and thus can indicate the stability of the vehicle for various parameters such as time, temperature, etc.

Ultrasound mediated drug delivery can be used with prototype acoustically active drug delivery vehicles. Acoustic parameters for radiation-force mediated concentration and disruption of acoustically-active lipospheres or AAL type vehicles can be optimized.

For in-vitro testing, a static assay chamber can be prepared containing a monolayer of A2085 human melanoma cells grown on Thermanox cover slips (Nalge Nunc, Rochester, N.Y.) along with a 1-ml volume of solution containing acoustically active delivery vehicles containing the fluorophore DiI as a model drug (concentration ˜1500 vehicles/microliter). Under static conditions, a very small population of the delivery vehicles was in contact with the cells. The static chamber can be mounted in a 500-ml polycarbonate tank with a 2.25 MHz spherically-focused transducer mounted in one wall of the tank such that its focus falls at the center of the cell monolayer in the assay chamber. Ultrasound pulse sequences can be generated with an arbitrary waveform generator and an RF amplifier. For testing, each plate can be exposed to the relevant pulse sequence for 3 min. After insonation, the chamber can be removed from the tank and disassembled, and the cover slips can be thoroughly rinsed and examined by fluorescence microscopy. The acoustic parameters can be optimized to localize drug delivery vehicles against the cell monolayer, cause vehicle disruption, and enhance delivery of the vehicle contents (such as a fluorophore) to the cell monolayer at the acoustic focus, without affecting cells outside of the acoustic focus. High magnification fluorescence microscopy after insonation reveals that the delivery vehicles fragment into sub-micron size fluorophore-bearing oil droplets which then adhered to the target cells.

Dynamic testing can be performed utilizing a 200 micron cellulose vessel. A solution of the delivery vehicles can be pumped through the tube at varying flow rates while ultrasound can be administered with a pulse sequence optimized to push the delivery vehicles against the vessel wall and disrupt them. Fluorescence microscopy reveals the ability to locally deliver a fluorophore to the vessel wall by concentration and disruption of the delivery vehicles. Fluorescence can be retained on the wall at shear values of 900 s-1. Additionally, ultrasound can be applied to selectively deliver a fluorophore. Tissue microvaculature can be perfused with a PBS solution containing acoustically-active drug delivery vehicles, and ultrasound can be applied with the concentration-disruption pulse sequence. Intravital fluorescence microscopy demonstrated the ability to concentrate the fluorophore DiI, the model drug, along the wall of vessels in the region of the acoustic focus.

One method for biodistribution imaging involves using optical probes, such as DiR, incorporated into the drug delivery vehicles, and imaging using a Xenogen IVIS100 optical imaging system. Biodistribution imaging has been performed with and without a tumor model. For the tumor model, prostate tumors can be imaged. Imaging studies can be performed as the tumors grow to a volume greater than 125 mm2 Contrast injection consisted of injecting approximately 50 to 100 microliters of the drug delivery agent through a 27 gauge vein catheter. In ultrasound studies, ultrasound can be generated by a 5 MHz 2″ spherically-focused ultrasound transducer. The transducer can be mounted in a custom water standoff, and positioned with a 3-axis positioning clamp. The transducer can be focused on the tumor region, and excited with an arbitrary waveform generator and RF amplifier.

Circulation time of microbubble vehicles with different shell characteristics can be studied. A 22-gauge catheter can be inserted into the vein for contrast agent injection. The transducer can be mechanically fixed in position with an articulated arm. Imaging can be done in B-mode to orient the imaging plane, and then the ultrasound system can be set to a contrast specific imaging sequence, Cadence™ Contrast Pulse Sequence (CPS) using a SONOLINE Antares™ ultrasound system (Siemens Medical Solutions USA, Inc., Ultrasound Division, Issaquah, Wash.) with a VF10-5 linear array transducer. Imaging can be done at 4.4 MHz, with 0.4% transmit power (M.I.=0.15), a gain of 38 dB, and a frame rate of 1 frame/sec. Low transmitting power and low frame rate were chosen to avoid microbubble destruction during imaging. Following recording of a pre-contrast baseline image, an intravenous injection of 250 μL of contrast agent solution can be provided at a constant rate over a 90-sec period. Starting with the commencement of the injection, CPS images can be recorded every 15 seconds for 15 minutes using an automated script and stored for offline analysis. In each case, detectable contrast had been cleared within 15 minutes. The ultrasound images can be analyzed offline using Image J (NIH, USA). A region of interest can be drawn and the mean pixel intensity for this area can be calculated. Background subtraction can be performed by subtracting the pre-contrast baseline image from each of the following post-contrast injection images. The mean image intensity can be normalized by the intensity at peak contrast enhancement, and the time for the signal intensity to decay by 50%, 20% and 10% can be determined for each contrast agent. Quantification of differences in circulation time due to microbubble resistance to the immune response for different microbubble types can be performed. Microbubbles with a double-layer PEG overbrush demonstrated significantly increased circulation time compared to standard agents.

A simultaneous optical-acoustical system can be utilized for visualizing the effects of ultrasound on the acoustically-active vehicles. Parameters for achieving maximum radiation-force induced concentration without disruption of the vehicles can be determined. An Olympus IX70 microscope interfaced with a high-speed video camera (Photron APX-RS) allows for observation and recording of micron-sized agents inside an optically transparent vessel. An ultrasonic transducer can be positioned such that its acoustic focus overlaps with the optical focus of the microscope. Transducers can consist of spherically-focused single-element transducers. 2.25 MHz (Panametrics V305) and 5 MHz (Panametrics V309) transducers can be used, as the resonance frequency of 3-6 micron delivery vehicles can fall within this range, however, other transducers can be used to adjust the frequency range. The 2.25 MHz transducer has a −6 dB bandwidth of 1.5-3.3 MHz, a focal length of 2″, and an aperture size of 0.75″. The 5 MHz transducer had a −6 dB bandwidth of 3.7-7.6 MHz, a focal length of 2″, and an aperture size of 0.5″. An arbitrary waveform generator (AWG 2021, Tektronix, Inc.) can be used to produce the excitation waveform. The waveform generator output can be amplified approximately 55 dB with a radio frequency power amplifier (3200L, ENI) to energize the transducer. In order to quantify parameters which produce optimal radiation-force induced concentration of delivery vehicles, the rate of vehicle displacement from the flow stream to the side of the tube can be optically quantified. Frequency, and pulse repetition frequency can be varied to maximum optimal displacement. The acoustic pressure can also be varied, with the goal of achieving maximum displacement without disruption of the vehicles.

Acoustic pressure calibrations can be performed using a needle hydrophone (PZT-Z44-0200, Specialty Engineering Associates) and a preamplifier (A17 dB, Specialty Engineering Associates) connected to a digital oscilloscope (9350, LeCroy). For alignment and calibration, the 200 micron needle hydrophone tip can be placed in the optical field of view. The received ultrasound signal can be maximized by adjusting the position of the transducer with a 3 axis manipulator. Acoustically absorbent rubber can be used to line the back wall of the water tank to eliminate standing waves and minimize reflections. The same simultaneous optical-acoustical system described can be utilized for visualizing the disruption of acoustically-active vehicles by ultrasound. Consistent vehicle disruption at the lowest Mechanical Index possible can be sought. Disruption to produce sub-micron fragments can be optimized. The ability to disrupt the delivery vehicle with a burst of ultrasound can be quantified as a function of acoustic pressure, pulse length, and frequency.

Optical imaging of drug-delivery vehicles incorporating a long-wavelength fluorophore can be used to observe the biodistribution with and without ultrasound. Acoustically Active Delivery Vehicles can be loaded with 1,1′-dioctadecyl-3,3,3′,3′-tetramethylindotricarbocyanine iodide (‘DiR’; DiIC18(7)). DiR is a long-wavelength lipophilic tracer dye which can be purchased commercially from Molecular Probes (Now called Invitrogen, Carlsbad, Calif.). The long wavelength probe (emission near 800 nm) can be suitable for serial biodistribution imaging due to the relatively low absorbance and background fluorescence of the tissue at longer wavelengths. Additionally, the biodistribution of vehicles loaded with Oregon Green Paclitaxel (also from Molecular Probes), a fluorescent version of the chemotherapeutic paclitaxel can be used. Although the shorter wavelength of this probe reduces its appeal for whole-body imaging, biodistribution can be obtained by imaging individual organs. Injections of 50 microliters of the drug delivery vehicles can be given, and optical images can be recorded with a Xenogen IVIS 100 imaging system. For serial imaging, images can be recorded every 2 minutes for 20 minutes, and then every 5 minutes for the next 15 minutes. Procedures for optical imaging can be similar as described above. Concentration of acoustically-active delivery vehicles and site-specific release of contents can be performed by applying an ultrasonic transducer focused at the target site, and administering a pulse sequence designed for radiation force and vehicle disruption. Imaging can be performed before and immediately after ultrasound treatment.

A 22-gauge catheter can be inserted into the vein for drug delivery vehicle injection. A spherically-focused ultrasound piston transducer (Valpey Fisher, Inc., Hopkinton, Mass.) can be positioned in a 3-axis positioning clamp and focused. The transducer can be energized with the pulse sequence designed from prior optimization of acoustic parameters, utilizing equipment described previously. Optical imaging can be performed using an Xenogen IVIS100 Optical imaging system. Regions of interest from insonated and control areas can be selected offline, and delivery of fluorophore from drug-delivery vehicles can be quantified over time, from before the ultrasound to 24 hours post treatment. A 22-gauge catheter can be inserted into the vein for contrast agent injection. The transducer can be mechanically fixed in position with an articulated arm. Imaging in B-mode can be used to orient the imaging plane, and then the ultrasound system can be set to a contrast specific imaging sequence, Cadence™ Contrast Pulse Sequence (CPS) using a SONOLINE Antares™ ultrasound system (Siemens Medical Solutions USA, Inc.).

Under another embodiment of the present invention, a hybrid particle complex can serve as both a contrast agent and a drug delivery system. The hybrid particle complex may be manufactured using microfluidic flow-focusing devices and methods as described herein. Contrast agents, as stabilized gas microbubbles for ultrasound (US) or superparamagnetic iron oxide (SPIO) particles for magnetic resonance imaging (MRI) can be important tools for improving the visibility of blood vessels and tissue, allowing for the detection of organ functional abnormalities and a more definitive diagnosis of the presence and extent of disease. In addition, lipid-shelled microbubbles as acoustically-active lipospheres (AAL) or metal nanoparticles show promise for drug delivery applications. In this embodiment, the utility of AAL-based drug delivery devices is combined with SPIO-based contrast agents to provide a new class of hybrid particle that can be useful for multimodal imaging with both ultrasound and MRI. The hybrid particles can also offer the benefit of being effective drug delivery devices that are able to delivery drugs, therapeutics, and other medicaments to selected body tissues or organs for release. Low frequency radio waves that pass through the body harmlessly and heat the iron oxide particles can melt the AAL shell, thereby releasing the drug contents within. Unlike ultrasound which has depth of penetration issues, low frequency radio waves can safely reach all remote body tissues and organs. These hybrid particle complexes can be functionalized for targeted imaging and drug delivery. The AALs can be produced by a PDMS flow-focusing device, forcing a central stream of gas (nitrogen) and three (stabilizer, lipid, and oil) double-sided liquid sheath flows through a narrow 20 μm orifice. Attachment of the SPIO particles can be achieved at the outlet reservoir.

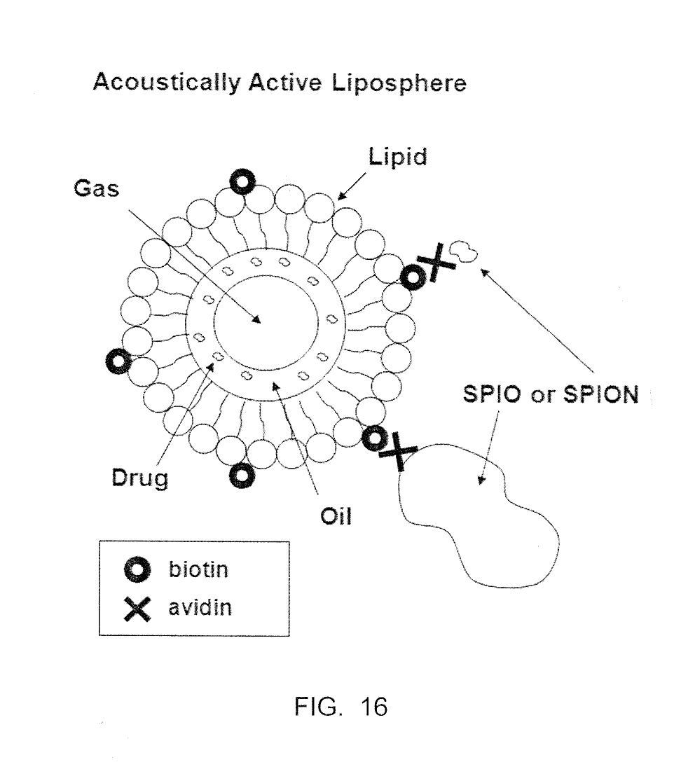

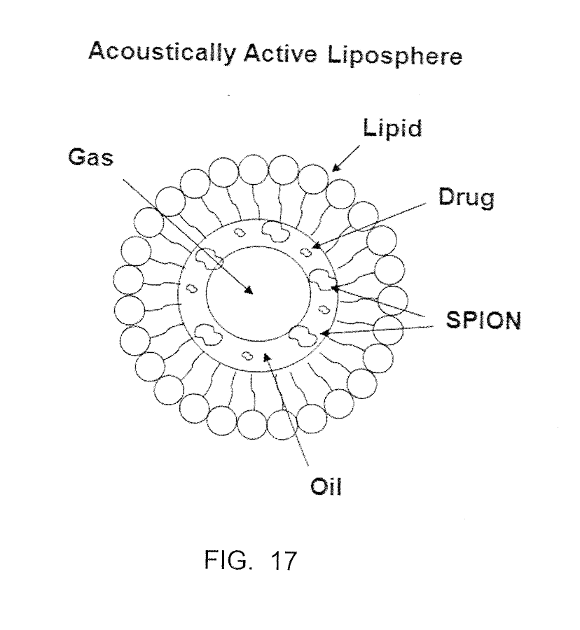

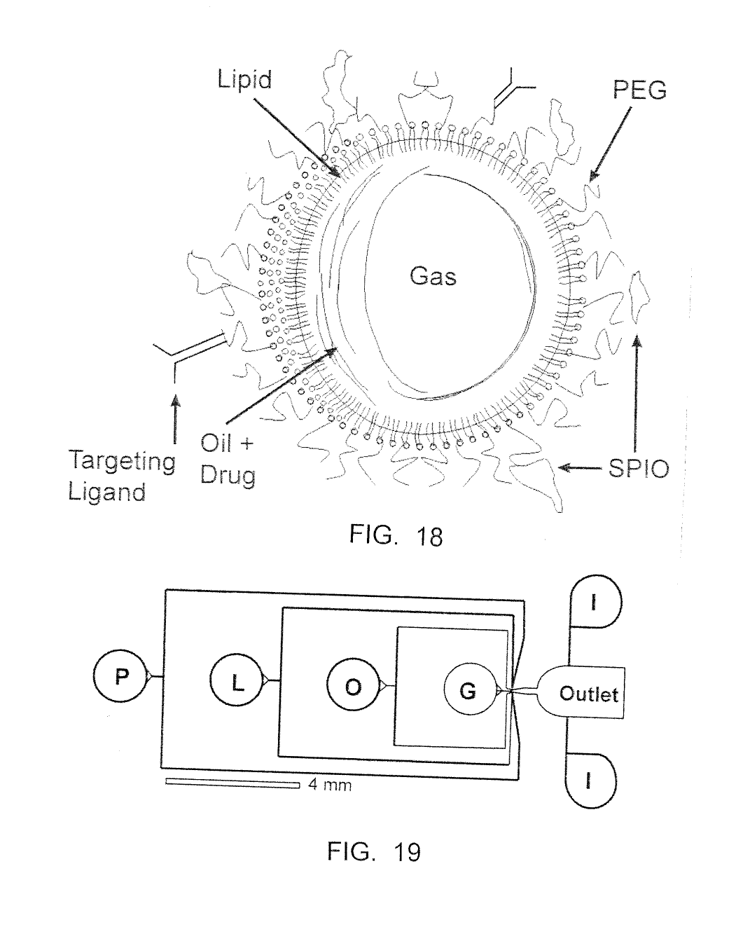

FIG. 16 is a diagram illustrating a hybrid particle complex in which superparamagnetic iron oxide particles (SPIO) and/or superparamagnetic iron oxide nano-particles (SPION) are attached to the exterior of an AAL. SPION refers to a smaller-sized version of SPIO particles. The AAL includes an interior gas region which makes the vehicle acoustically active to ultrasound pulses. The gas region is surrounded by an inner oily layer that contains the drug, therapeutic agent, or medicament which is dissolved therein. The outer lipid shell stabilizes the vehicle. FIG. 17 is a diagram illustrating an alternative configuration of a hybrid particle in which magnetic iron oxide particles are contained within the inner oily layer. FIG. 18 is a diagram illustrating a three dimensional cross-section of a hybrid particle complex that includes a targeting ligand on the exterior thereof. The targeting ligand is bound to the hybrid particle via a polyethylene glycol (PEG) layer. The targeting ligand may be custom designed to target particular diseased tissue such as cancer tissue. The targeting ligand may also target particular tissue or cell types.

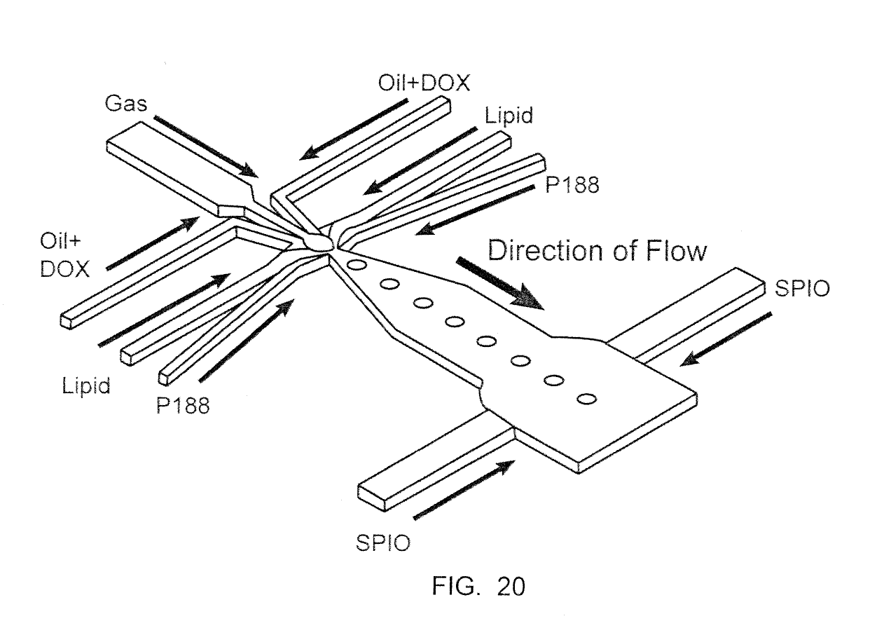

FIG. 19 is a diagram illustrating a top view of a complete microfluidic channel network that is used for hybrid particle complex production. All channels have a rectangular cross section and a height of 25 μm. The widths of the P188 P, lipid L, oil O, gas G, and iron oxide I channels are {20, 30, 25, 20, 50 μm} respectively, and the orifice size is 20 μm. Only the L and G inlets are required for standard lipid microbubble ultrasound contrast agent production. FIG. 20 is a diagram illustrating a magnified view of region of interest with arrows corresponding to flow direction.

Microfluidic channels can be used to spatially organize the various fluid streams containing various components of the hybrid particle complex and direct them to a focusing region to promote rapid interdiffusion and self-assembly. The microfluidic device, can be manufactured in poly(dimethylsiloxane) (PDMS) by soft lithography as described herein, can combine three hydrodynamic flow-focusing regions together and feature expanding nozzle geometry to create a point of highest shear force. Such a shear-induced break-off of fluid streams at the narrowest point (orifice) enables precise control of particle sizes. Combining the three hydrodynamic flow-focusing geometries together into a single region can be less sensitive to gas pressure than in double or triple flow-focusing geometry where the flow-focusing regions are separated by a specific distance. This can be critical to the stable formation of smaller sized multi-layer gas lipospheres, since liposphere size and production rate are highly prone to downstream pressure conditions. The shear-induced break off at the orifice results in the self-assembly of phospholipids to a lipid layer around the gas and oil phases. Subsequent attachment to SPIO particles occurs downstream at the outlet reservoir via the avidin/biotin interaction, producing the final hybrid particle complex.

The hybrid particle complexes are formed by forcing a central stream of nitrogen gas along with three (stabilizer, lipid, and oil) double-sided liquid sheath flows through a narrow 20 micron orifice. Attachment to metal containing substance, metal oxide or magnetic iron oxide particles can be achieved at the outlet reservoir. The outermost sheath flow stream can contain a 10% Poloxamer 188 solution (e.g., surfactant) that enhances the stability of the AAL shell. In particular, the 10% Poloxamer 188 solution prevents AALs from combining with one another. The middle sheath flow stream can contain a 10% aqueous glycerol/propylene glycol mixture with the stabilizing lipids DSPC (1,2-distearoyl-sn-glycero-3-phosphocholine, Avanti Polar Lipids) and DSPE-PEG2000-Biotin (1,2-Distearoyl-sn-Glycero-3-Phosphoethanolamine-N-(Biotinyl(Polyethylene Glycol)2000), Avanti Polar Lipids) at a 9:1 molar ratio and concentration of 0.5 mg/mL DSPC. The inner sheath flow stream can consist of triacetin oil (Glyceryl triacetate, Sigma) that contains the drug, therapeutic agent, or medicament dissolved or entrained therein.

Oil Blue N dye from Sigma (St Louis, Mo.) can be premixed at a concentration of 0.01 mg mL⁻¹ with the triacetin oil for easier visualization. In addition, Doxorubicin (DOX) from Sigma (St Louis, Mo.) can be used as the model antitumor drug due to the intrinsic fluorescence properties of the molecule, and can be mixed with triacetin at a concentration of 1 mmol/L. It should be understood that that other drugs, therapeutic agents, medicaments and the like may be mixed with the oil phase. Irregularly shaped superparamagnetic iron oxide particles can be covered with a Streptavidin coating for attachment to biotin groups on the AAL shell can be used to create the hybrid complexes. Irregularly shaped streptavidin coated SPIO particles (BM551, Bangs Laboratories), approximately 1.5 μm in diameter can be used to create complexes. The suspension is supplied in phosphate buffered saline (PBS) solution (pH 7.5) containing 0.1% BSA with EDTA and sodium azide added. The original particle concentration is 5 mg mL⁻¹ and is further diluted to 1 mg mL⁻¹. The SPIO particles, approximately 2 μm in size, are >90% in iron oxide composition. Streptavidin is covalently attached to the SPIO surface, with each molecule providing four binding sites for the biotin groups of the AAL shell. For direct binding to cancer cells, biotin (EZ-Link Sulfo-NHS-LC-Biotin, Pierce) can be used for labeling cell surface proteins.

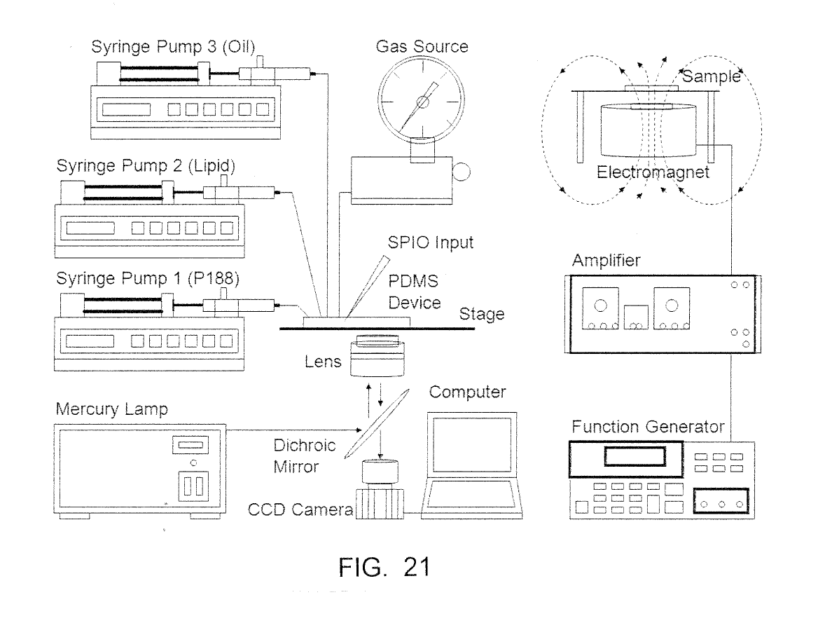

FIG. 21 is a diagram illustrating a setup used to obtain images of the Oil Blue N dye liberated from the hybrid complexes in response to applied radiofrequency energy. Nitrogen (N2, Airgas) gas is supplied from a pressurized tank via flexible tubing and delivered into the chamber using a micro flow meter consisting of a high-accuracy filled pressure gauge (EW-68022-02, Cole-Parmer Instrument Company) coupled by a three-way pressure gauge tee to a micro-metering needle valve assembly (P-445, Upchurch Scientific). The continuous liquid phase mixtures can be pumped in 3 cc Becton-Dickinson syringes and can be pumped into the microfluidic device via 23 gauge hypodermic tubing at constant flow rates using three digitally controlled syringe pumps (Pico Plus, Harvard Apparatus). A 200 mL pipette can be used to introduce the suspension of SPIO particles at the outlet. An inverted microscope (TE2000, Nikon) and high-speed camera (Fastcam PCI-10K, Photron Ltd.) can be used to capture images and record movies. A function generator and power amplifier can be used to drive a standard AC electromagnet, which energizes the iron oxide particles. Sample temperature is measured with a laser infrared interferometer.

A file viewer (PFV, Photron Ltd.) and image analysis program (ImageJ, NIH) can be used for data processing and measurements. The polydispersity index σ=δ/_{davg}×100% was calculated from the average vehicle size davg and standard deviation δ, determined by measuring the sizes of AALs from recorded images.

Hybrid complexes in the form of a foam can be collected using a glass pipette from the outlet reservoir. Aliquots can be transferred onto glass slides and secured with plastic cover slips to reduce fluid flow. The sample can be positioned 5 mm over a standard AC electromagnet (22 mm (H)×38 mm (W), 246 ohm), driven by an arbitrary function generator (AFG320, Tektronix) and power amplifier (Model 7500, Krohn-Hite). This configuration enables the monitoring of sample movement and drug release by energizing the SPIO with alternating magnetic fields. The movement of individual hybrid complexes can be imaged in brightfield (optical magnification 10×) under an upright microscope (Eclipse L150, Nikon) with a high resolution color CCD camera (Insight Spot QE, Diagnostic Instruments). By measurements of time taken to move a known distance, the velocity of the hybrid complexes can be calculated. Bulk sample temperature can be measured with an Extech (Waltham, Mass.) laser infrared interferometer and field strength with an AlphaLab (Salt Lake City, Utah) AC/DC magnetometer.

A 15 micron hybrid vehicle with attached brown irregular shaped magnetic iron oxide particles (˜2 μm) can be produced. The oily blue layer containing doxorubicin (DOX) can be visible within the black lipid layer. At specific flow rates (QP188=10 μL/min, Qlipid=20 μL/min, Qoil=1 μL/min, Pgas=1.5 psi), stable and monodisperse AALs can be produced with attached iron oxide particles. The oil layer can be visible encapsulated within the black lipid shell. DOX fluorescence at the interior lipid-oil interface can be visible. Broken DSPC lipid shells at the chain melting (gel to liquid-crystalline) temperature of 55° C. can be visible. DOX fluorescence indicating local release at 55° C. can be visible.

The physical principles of magnetophoresis are derived from the magnetic force exerted on a superparamagnetic particle at a distance by a magnetic field gradient. For a one-dimensional magnetic field gradient, the total magnetic force Fₜₛₚₘ balances the viscous (stokes) drag F_{D} for an SPIO-AAL complex moving in a fluid due to a magnetic field gradient, as represented by the following equation:

$${F}_{D} = - 6 π {R}_{a} η v = - {N}_{spm} \frac{1}{2} \frac{{V}_{spm} {χ}_{spm}}{{μ}_{0}} ∇ {B}^{2} = - {F}_{tspm}$$

where Rₐ is the radius of the AAL, η is the viscosity of the aqueous medium, v is the velocity of the AAL, Nₛₚₘ is the number of SPIO particles conjugated to an AAL, Vₛₚₘ is the volume of the SPIO particle, χₛₚₘ is the net magnetic susceptibility of a SPIO particle in aqueous solution, B is the magnetic field, and μ₀ is the permeability of free space. The procedure for catabolism of tumors involves dispersing magnetic particles throughout the target tissue, and then applying an AC magnetic field of sufficient strength and frequency to cause the particles to heat. This thermal triggering can also be used to release a drug or payload from the vehicle.

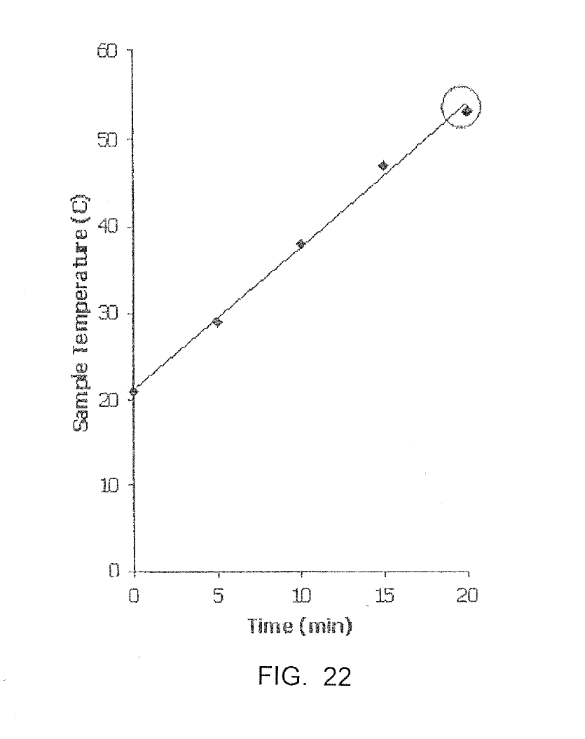

The velocity v of the hybrid particle complex in the aqueous medium can be calculated from the above equation. The experimentally calculated complex velocity is 0.12 mm/s, but can be orders of magnitude different according to theory, depending on the number of SPIO attached to AALs, irregularity in SPIO shape, solution viscosity changes due to oil, and strength of the external magnetic field. Application of alternating magnetic fields at a maximum strength of 775 Gauss increases sample temperature to 55° C. in 10 minutes by way of heating the iron oxide particles, melting the lipids and releasing DOX. FIG. 22 is a graph illustrating sample temperature as a function of time in response to application of a 775 Gauss alternating magnetic field.

The main advantage to the hybrid complexes is that detection and therapy will be integrated. Early diagnosis of cancer improves prognosis, and multimodal hybrid particle complexes will allow clinicians to diagnose cancer and other diseases in their earliest stages by providing more comprehensive data with different medial imaging techniques (multimodal imaging). The flexibility of the lipid shell components allow for a wide variety of targeting moieties to be incorporated, allowing for selective targeting of specific biological sites. Molecular and morphological changes at the cellular level can be addressed, and clinicians will be able to precisely monitor and track drug action in real-time. Patients will also not have to come in for imaging, then chemotherapy, and then repeated scans.

The hybrid particle complexes can serve as a contrast agent for both ultrasound and MRI, and the AAL composition makes them able to carry a large and precise drug payload to the target site. They are ultrasonically active and can be steered and directed to target sites by the acoustic radiation force, or can also be localized by magnetic fields due to the SPIO. Either the acoustic radiation force or heating the SPIOs with low frequency radio waves can be used to release drug contents.

Typically an external device may be used to deliver the ultrasound or low frequency radio waves to the subject. The device may deliver ultrasound or radiofrequency radiation to the subject in a targeted or non-targeted manner. For instance, the entire body of the subject may be subject to ultrasound or radio frequency energy. Alternatively, ultrasound or radio frequency energy may be focused to one or more body regions of the patient (e.g., targeted to one organ or the like). The ultrasound or radio frequency energy may be introduced internally through an internal probe or the like that can be percutaneously advanced into a subject. The external device may be hand held or larger such as a full MRI machine.

The hybrid particle complexes can serve as a contrast agent for both ultrasound and MRI, and the AAL composition makes them able to carry a large and precise drug payload to the target site. Challenging hydrophobic drugs such as paclitaxel can be dissolved at high concentrations (70 mg/ml) in the lipid-oil complex of the AALs. The microfluidic production method will enable more precise dosages of drug to be delivered. The hybrid complexes are solid at body temperature, but meltable with heat. The SPIO component can be remotely actuated by EMF activation and the AAL consists of a composition with known acoustic activity to ultrasound radiation force. The controlled production of these hybrid complexes provides exciting opportunities for in-vivo medical research (e.g. cancer), and demonstrates the versatility of the microfluidic platform in creating multifunctional particles. Diagnostics and therapeutics no longer have to be sequential elements in patient care. As contrast agents, these particles can improve disease diagnosis. As a drug delivery system, these particles will be able to minimize non-specific drug-carrier accumulation and reduce the toxicity involved with most chemotherapeutic agents.

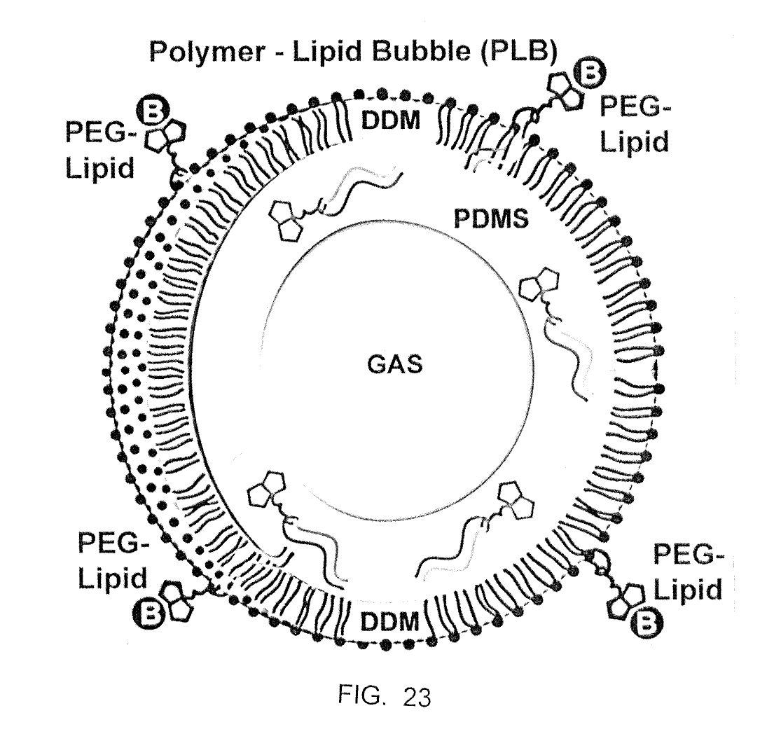

FIG. 23 is a diagram illustrating a hybrid polymer-lipid functionalized microbubble of one embodiment of the present invention. Under this embodiment of the present invention, the elasticity of PDMS can be combined with the functional characteristics of polyethyleneglycol (PEG)-lipid derivatives to create a new class of stable and compressible polymer microbubbles as illustrated in FIG. 23. The outer PDMS-lipid shell stabilizes the vehicle and the gas interior makes the vehicle compressible.

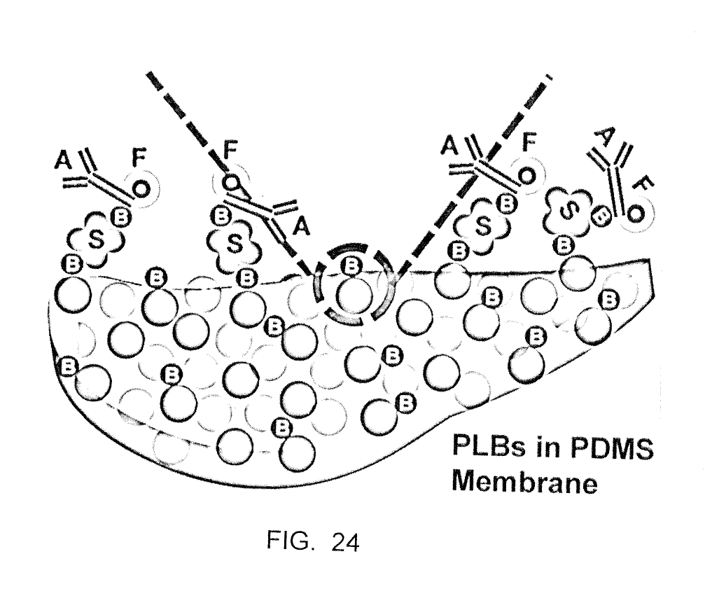

FIG. 24 is a diagram illustrating cured spherical PLBs that are densely or loosely packed within porous elastic membrane for biosensing of one embodiment of the present invention. B, S, A, and F correspond to Biotin, Streptavidin, Antibody, and Fluorophore respectively. Particle trapping within microchannels is possible for biosensing applications as illustrated in FIG. 24, and their use as a template material in the production of 3-D porous structures and surfaces. Incorporating PDMS as a shell component improves the functionality and stability (>1 month) of the hybrid particles due to the thermally maneuverable solidification process. Some hybrid particles have been observed as stable over a six month period of time. With a gas core the particles are compressible. Furthermore they serve as a non-sacrificial template material for creating porous elastomers, requiring no cumbersome post-processing removal steps. Adding PEG-lipid components within the PDMS shell enables a wide variety of targeting moieties to be incorporated (avidin-biotin, antibodies, DNA, magnetic particles, etc.), allowing for selective targeting. A biocompatible PDMS-lipid-gas composition offers opportunities in creating porous engineered scaffolds, acoustically-responsive delivery (i.e. drug) agents, and bio-based packaging materials.

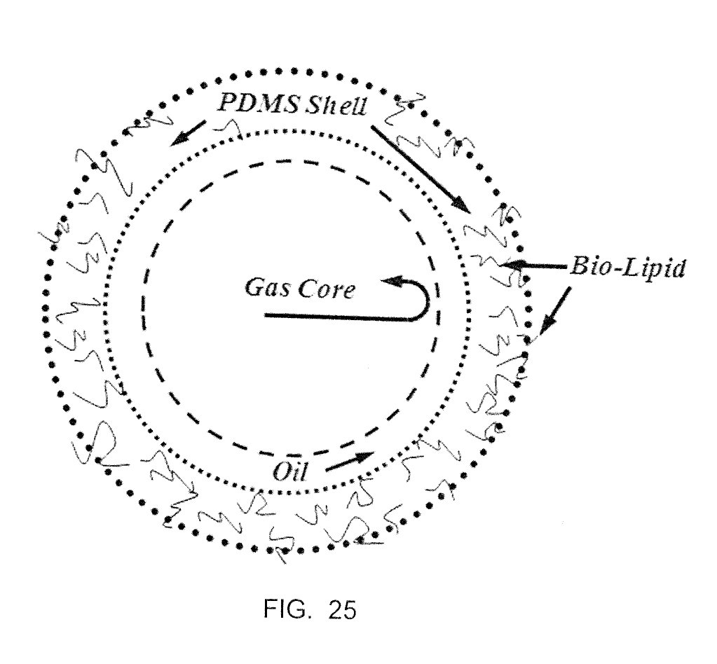

FIG. 25 is a diagram illustrating a PDMS-Schematic of a PDMS-lipid functionalized drug delivery microbubble of one embodiment of the present invention. The outer PDMS-lipid shell stabilizes the vehicle and the inner oil layer can contain dissolved therapeutics. The gas interior makes the vehicle compressible and acoustically active to ultrasound pulses.

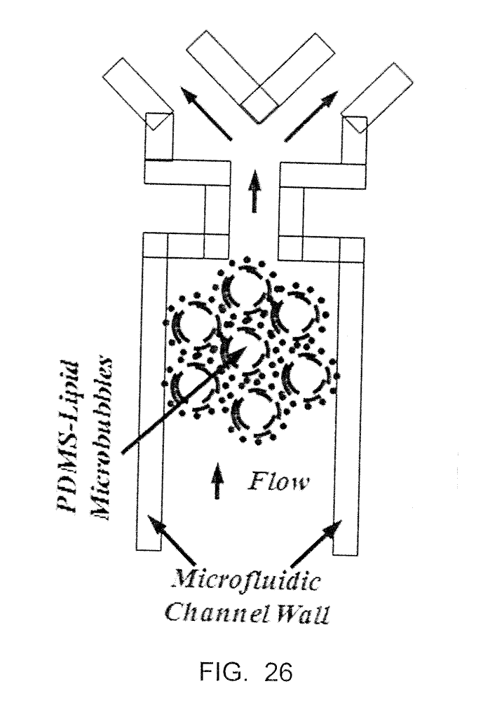

FIG. 26 is a diagram illustrating how cured spherical PDMS microbubbles can be densely or loosely packed on-chip based on microfluidic chip geometry at specific locations for biodetection applications of one embodiment of the present invention.

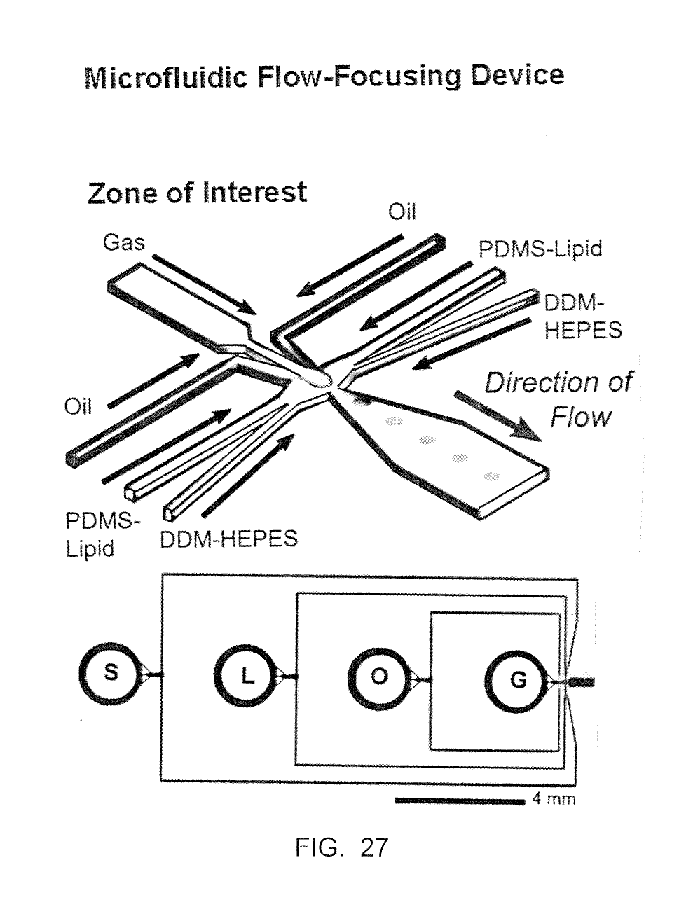

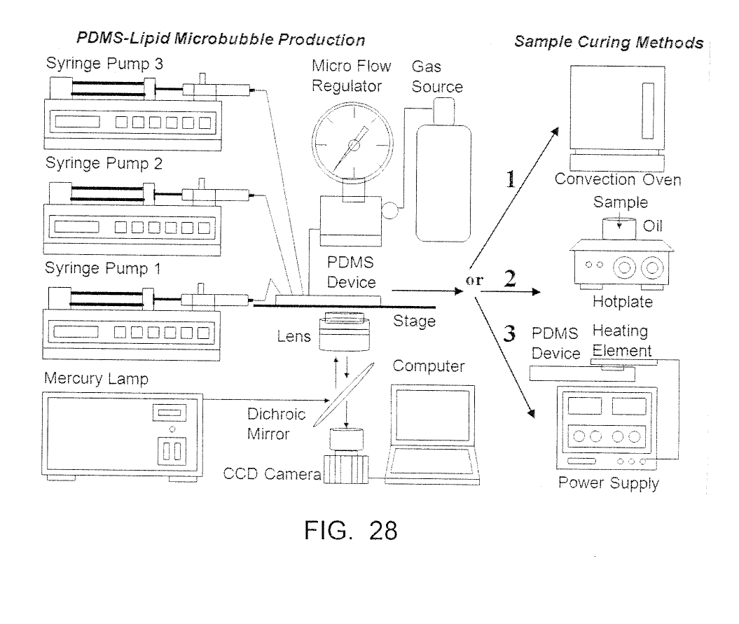

FIG. 27 is a diagram illustrating the geometry of complete device and zone of interest for particle production where S, L, O, and G correspond to the DDM-HEPES, PDMS-lipid, oil, and gas inlets respectively of one embodiment of the present invention. The optional oil inlet allows for multi-layer particle production. All channels have a rectangular cross section and a height of 25-45 μm. The widths of the outer solvent, lipid, oil, and gas inlet channels are {20, 30, 20, 20 μm} respectively. The device features an expanding nozzle with an orifice width of 20 μm. PLB generation can occur at the orifice. Particles move within the channel and rise to the top of the outlet.

The multiphase flow-focusing platform (illustrated in FIG. 27) is used to spatially organize fluid streams containing various components of hybrid microbubbles (stabilizer, PDMS-lipid, oil (optional), and gas) and direct them to a shear-induced break-off point (orifice), enabling precise control of fluid emulsion sizes. These fluid segments rapidly acquire a spherical shape due to the action of interfacial tension. A HEPES-buffered saline solution containing 1% n-Dodecyl β-D-maltoside (DDM) can serve as the outermost flow stream. As a non-ionic surfactant, DDM has dual hydrophobic/hydrophilic properties that allow lipid displacement, while still providing coverage on the native lipid-modified PDMS shell. Considerable reduction in pre-cure PDMS (Sylgard 184) viscosity (3900 cPs) to a range between 100-500 cPs can be necessary prior to use of the mixture as the primary continuous phase within microchannels. PDMS oligomers can be diluted with hexane and low viscosity (4.5 cPs) silicon oil at equal ratios to approximately 70% by weight. Dilution with hexane produces the least viscous solution, but 4.5 cPs silicon oil can produce a fast cure time while still providing a workable solution during the experiment time of 2 hrs. The final combination (160 cPs) can be empirically determined. Due to chloroform solubility in hexane, approximately 50 μL of the lipid 1,2-Distearoyl-sn-Glycero-3-Phosphoethanolamine-N-(Biotinyl(PolyethyleneGlycol)2000) (DSPE-PEG2000-Biotin) can be added to the solvent mixture at a concentration of 5.0 mg mL⁻¹. The diluted PDMS-lipid mixture can serve as the inner sheath flow stream.

The PDMS microbubbles can be produced by a microfluidic flow-focusing device as described herein, forcing a central stream of nitrogen gas and multiple liquid sheath flows through a narrow 20 micron orifice. Washing the microbubbles in a heated mineral oil bath at 40-70° C. enables controlled solidification of the particles themselves. As drug delivery vehicles, further heating beyond the lipid chain-melting temperature can destabilize the shell, releasing encapsulated contents into the surroundings.

FIG. 28 is a diagram illustrating a setup of a flow-focusing device attached to fluid sources on a microscope imaging system and a setup of three sample curing methods of one embodiment of the present invention. In one method (top) the sample is cured in an oven. In another method (middle) the sample is cured on a hotplate. In another method (bottom) the sample is cured in the microfluidic device (e.g., PDMS device) using a heating element coupled to a power supply. The heating element heats the oil contained within the outlet reservoir. Referring to FIG. 28, gas is supplied from a pressurized tank via flexible tubing and delivered into the chamber using a micro flow meter. The continuous liquid phase mixtures are pumped at constant flow rates using digitally controlled syringe pumps. An inverted microscope and high-speed camera are used to capture images and record movies. At a specific range of flow rates (Q_{DDM-outer}=5-15 μL/min, Q_{PDMS-liqid}=1-10 μL/min, Qₒᵢₗ₋ᵢₙₙₑᵣ=1.0-0.5 μL/min, P_{gas}=1.0-5.0 psi) stable and monodisperse elastic gas-filled lipospheres can be produced. Washing the microbubbles in a heated mineral oil bath between 40° C.-70° C. enables controlled solidification of the particles themselves. Sample temperature can be measured with a laser infrared interferometer. A heating element can be incorporated on-chip, allowing for control of vehicle curing within the microfluidic device.

The stabilizing gas octafluorocyclobutane (C₄F₈) can be used as the dispersed phase. The diameter does not change significantly for these vehicles as they rise from the outlet due to buoyancy. In other embodiments gases such as nitrogen, carbon dioxide, perfluorocarbons, or mixtures may also be used. Stable production rates of 1.5×103 PLBs per minute have been achieved with runtimes lasting several hours. PLBs can be visible in Brightfield, collected on the cover glass after approximately 10 min of 120° C. heat treatment. The outer PDMS-lipid shell with DMM stabilizes the vehicle, and incorporates biotin-PEG for biosensing and targeting. With a hollow gas core, the particles are compressible. The orange-red fluorescence of DiIC18, a lipophilic membrane stain that is weakly fluorescent until incorporated into membranes, confirms the lipid coating within the PDMS shell.

The same microfluidic device may be used for encapsulating materials or drugs within a PLB. For producing a drug delivery vehicle in the form of a 30 micron PDMS-lipid multi-layer microbubble, the inner sheath flow stream consists of triacetin oil, premixed with Oil Blue N dye for easier visualization. The additional triacetin oil layer is capable of carrying chemotherapeutics. The volume fraction of the oil layer can be adjusted using flow rates. Doxorubicin (DOX) can be used as the model antitumor drug due to the intrinsic fluorescence properties of the molecule, and is mixed with triacetin at a concentration of μmol/L. Streptavidin-bound superparamagnetic iron oxide particles (approximately 1 micron) can be attached to the biotinylated lipid of the PDMS shell of 30 micron PDMS-lipid microbubbles. Radio waves that heat only the iron oxide particles can potentially melt the outer shell, releasing the drug into the surroundings.

PDMS-lipid microbubbles can be trapped within narrow microchannels of a microfluidic device. Cured PDMS-lipid microbubbles can be densely or loosely packed on-chip based on microfluidic trapping geometries at permanent locations. Microfluidic channel geometries can be designed based on flow resistances to hold particles in place. Large 50 micro PDMS-lipid microbubbles can also be produced. PLBs can also be linked together after heat treatment to form a chain of particles. 10 um PLBs can be trapped within microchannels of a microfluidic device. 45 um PLBs can be trapped within microchannels of a microfluidic device. Fluid continues to flow. PLBs can be embedded within PDMS elastomer matrix after a 120 degree C. heat treatment. Trapped PLBs can be on-chip and within porous elastomers. Two sizes of PLBs (10 μm and 45 μm) were captured and immobilized in microfluidic traps to demonstrate potential biosensing applications on-chip while fluid continues to move. When using 4.5 cPs silicon oil without further dilution, heating the sample suspension in a convection oven at 120° C. for 10 min solidifies the entire sample, embedding the microbubbles within the polymer matrix. Spreading this outlet solution with a glass stir rod and subsequent heating allows creation of thin (−50 μm thick) porous elastomeric membranes. Micron sized holes can be produced. These pores become a permanent feature of the polymer matrix. After thermal solidification, they are size-stable for more than six months of monitoring.

The packing density can largely be controlled based on production rate, resulting in densely packed PLB clusters bound to a thin elastomer layer. Transferring the sample to a heated oil bath enables controlled solidification of the particles themselves. At 120° C., PLBs become rapidly embedded in the matrix in a matter of seconds, even with continuous stirring of the oil during the solidification process. With 40° C. curing, free floating single layer PLBs can be obtained isolated and separated from the PDMS carrier fluid.

DSPE-PEG2000-Biotin can be chosen to test the functionalization of the PDMS microbubbles, and potential for biosensing applications. Similar PEG-lipid derivatives can be used to prolong the in-vivo circulation time of liposomes and to evaluate stearic stabilization of amphipathic polymers. After observing clusters in Brightfield, fluorescence imaging with biotinylated Alexa Fluor 488 labeled goat anti-mouse isotype-specific IgG antibodies can be performed to visualize the shell surface. DDM can minimize nonspecific protein adsorption on PDMS surfaces to the single molecule level. The inhomogeneity of the bright green fluorescence indicates that DDM may restrict protein adsorption on the PLB shell and IgG antibody binding to functionalized outer PLB shell. More importantly, these results indicate that biotin groups are present on the outer PLB shell surface after PEG-lipid modification, and their presence allows avidin binding. No decrease in shell surface fluorescence intensity is observed after sample washing, or after a few days at STP conditions

HT-1080 human fibrosarcoma-derived cells were donated, and can be obtained from the American Type Culture Collection. Cells can be cultured in DMEM (Dulbecco's modified Eagle's medium, Invitrogen) containing 10% FBS (fetal bovine serum, Invitrogen). PDMS hybrid microbubbles can be prepared as described earlier, with the addition of 50 μL DSPS (1,2-distearoyl-sn-glycero-3-phospho-L-serine, Avanti Polar Lipids) to the solution at 5 mg mL⁻¹. Thin membranes of PDMS-lipid microbubbles can be obtained on glass slides. Attachment and uptake of microbubbles with HT-1080 fibrosarcoma cells can be done in the presence of serum, underneath a cover slip. Cells favorably attach to <15 μm PDMS microbubble lipospheres. Surface phosphatidylserine (PS) can be confirmed by determining the ability of the cells to bind to the microbubbles. The cells would otherwise clump together in clusters. It is possible that the serum provides additional molecules to promote adhesion, or may stimulate uptake by phagocytes.

The following chemicals and methods were used to construct and analyze the PLBs.

Polydimethylsiloxane (PDMS) prepolymer (Sylgard 184) can be purchased from K. R. Anderson (Morgan Hill, Calif.). The stabilizing lipid DSPE-PEG2000-Biotin (1,2-Distearoyl-sn-Glycero-3-Phosphoethanolamine-N-(Biotinyl(Polyethylene Glycol)2000) can be purchased from Avanti Polar Lipids (Alabaster, Ala.) as a dry powder. N-Dodecyl β-D-maltoside (DDM), HEPES buffered saline, Hexane, Silicone Oil (Dow Corning Corporation 200 fluid), Triacetin (Glyceryl triacetate), and Oil Blue N dye can be purchased from Sigma (St Louis, Mo.). DiI-C18 (1,1′-dioctadecyl-3,3,3′,3′-tetramethylindocarbocyanine perchlorate), Alexa Fluor® 488 goat anti-mouse IgG, and Phosphate-buffered saline (PBS) are purchased from Invitrogen (Carlsbad, Calif.). EZ-Link Sulfo-NHS-LC-Biotin (sulfosuccinimidyl-6-(biotinamido)hexanoate) and ImmunoPure Avidin can be purchased from Pierce (Rockford, Ill.).

The microfluidic devices can be fabricated from a SU-8 (MicroChem) master with the channels molded in PDMS using standard soft lithography and rapid prototyping techniques. Fluidic channel geometries can be designed in Illustrator (Adobe, Inc.) and printed at 20,000 dpi onto transparency masks by CAD/Art Services (Bandon, Oreg.). In a clean room, a 3 in. silicon wafer can be spin-coated at 3000 rpm with a UV-curable epoxy (SU-8-25, MicroChem) to form a 25-45 μm high layer. Exposure to UV light through the photomask containing the channel pattern cross-links the exposed areas of SU-8, which then remain on the wafer after development.

The wafer can be used to cast a replica in the silicone elastomer polydimethylsiloxane (PDMS) (Sylgard 184, Dow Corning), consisting of a 10:1 prepolymer and curing agent ratio. The prepared mixture of PDMS can be degassed under vacuum, poured onto the wafer with template, and cured for at least 4 hrs at 65° C. in a dry oven. The cured PDMS can be peeled from the wafer in a laminar flow hood. Inlets and outlets were punched with a blunt 18 G needle, and the stamp can be bonded to clean soda lime glass (Corning) after 90 s of air plasma (200 mTorr, 200 W) treatment with an expanded air plasma cleaner (Harrick Scientific, N.Y.).

A 1% n-Dodecyl β-D-maltoside (DDM) HEPES-buffered saline solution can be used without further dilution for the outermost sheath flow stream. For the inner sheath flow stream, PDMS oligomers (20:1 of base and catalyst) can be diluted with hexane and low viscosity (4.5 cPs) silicon oil to approximately 70% by weight at a 1:1 ratio of hexane to silicon oil. 50 μL of the lipid DSPE-PEG2000-Biotin in chloroform can be added to the PDMS-solvent mixture at a concentration of 5.0 mg mL⁻¹.

Solvent Properties

| | | | | | Sylgard 184 |
|---|---|---|---|---|---|
| | Hexane | Toluene | Si Oil-1 | Si Oil-2 | (pre-cure) |
| Density (g | 0.672 | 0.865 | 0.913 | 0.960 | 1.03 |
| mL⁻¹) | | | | | |
| Viscosity (cPs) | 0.308 | 0.590 | 4.5 | 500 | 3900 |
| at 20° C. | | | | | |

PDMS-Solvent Mixtures & Curing Time at 120° C.

| | PDMS-Solvent Cure % | | | | |
|---|---|---|---|---|---|
| Time (min) | Hexane | Toluene | Si Oil-1 | Si Oil-2 | 1:1 Hexane Si-Oil |
| 5 | 15% | 20% | 50% | 90% | 30% |
| 10 | 75% | 80% | 95% | 100% | 90% |
| 15 | 100% | 100% | 100% | 100% | 100% |

The PDMS microfluidic device can be placed on an inverted fluorescence microscope (TE2000, Nikon) and a high-speed camera (Fastcam PCI-10K, Photron Ltd.) used to capture monochrome still images and record movies. Octafluorocyclobutane (C₄F₈, Scott Specialty Gases) can be supplied from a pressurized tank via clear Tygon® tubing and delivered into the gas inlet of the microfluidic chamber using a homemade micro flow meter. The continuous liquid phase mixtures are pumped into the microfluidic device using two digitally controlled syringe pumps (Pico Plus, Harvard Apparatus). A file viewer (PFV, Photron Ltd.) and image analysis program (ImageJ, NIH) are used for data processing and measurements. The polydispersity index σ=δ/d_{avg}×100% can be calculated from the average vehicle size davg and standard deviation δ, determined by measuring the sizes of particles from recorded images.

PDMS-lipid microbubbles can be trapped within microchannels, allowed to collect on the glass slide, or transferred in the form of a foam using Teflon® tubing connected from the outlet. A hotplate (Corning) or convection oven at 120° C. solidifies the sample in 10 min. Washing the PDMS-lipid microbubbles in a heated mineral oil bath at 40° C. enables controlled solidification of the particles themselves. Bulk sample temperature can be measured with an Extech (Waltham, Mass.) laser infrared interferometer.

Approximately 5 μg of the fluorescent probe DiIC18 can be added to the initial chloroform lipid suspension and sonicated for 20 min at room temperature. The Alexa Fluor® labeled goat anti-mouse isotype-specific IgG antibodies are supplied as 2 mg mL⁻¹ solutions in 0.1 M sodium phosphate, 0.1 M NaCl, pH 7.5, containing 5 mM sodium azide. For biotinylating the proteins in solution, a 20-fold molar excess of biotin reagent can be used to label 1 mg of antibody. The reaction is incubated at room temperature for 30 min. Unlabeled avidin solutions at 10 mg mL⁻¹ can be prepared by dissolving the powder in PBS. For PDMS microbubble liposphere binding studies, 10 μl of avidin can be added to 500 μl of biotinylated particles. After a 5 min wait time, excess reagents can be removed by washing with DI water. A 25 μl solution of antibody can then be washed over 1 mL volume of PDMS-lipid microbubbles and rinsed with DI water after a 5 min wait time.

The specimen can be imaged with a Zeiss EVO LS 15 environmental scanning electron microscope (Carl Zeiss SMT AG, Germany) using 2.0 kV accelerating voltage at a 10 mm working distance. Fluorescence imaging can be performed with an upright microscope (Eclipse E800, Nikon) with Nikon 10×NA 0.3 and 40×NA 0.75 DIC objectives. The sample can be positioned on the stage and illuminated by a 100 W mercury lamp (Chiu Technical Corp) using optical filters for illumination with blue (λ=475-490 nm, FITC) or green (λ=505-535 nm, TRITC) light depending on fluorophore. Detection of green fluorescence occurred at λ=519 nm due to Alexa Fluor® 488 labeling and orange-red fluorescence occurred at λ=569 nm due to DiI labeling. Images can be recorded with a 2-Megapixel color CCD camera (MicroFIRE, Olympus) and processed with PictureFrame 2.0 (Optronics) software.

The synthesis of long-lasting polymer-lipid microbubbles with a microfluidic method and their capability in biodetection and the creation of porous structures is described. After thermal solidification, they are highly size-stable and have low polydispersity values (σ<10%). By trapping the particles on-chip, multiple detection schemes may be incorporated based on the chemistry of the PEG-lipid derivatives in the PDMS shell. With a densely packed arrangement, they may serve as acoustic sources when energized with ultrasound radiation force.

The use of PLBs may have particular applications in point-of-care diagnostics and as bio-based packaging materials to improve the quality and safety of foods. Potential in-vivo applications exist for use as porous engineered scaffolds and as vehicles for targeted therapeutics.

The possible uses of PDMS microbubbles include stand-alone applications for imaging, drug delivery, and biodetection, but also off-chip as a template material for creating microfluidic “lab-on-a-chip” components with varying composition. From a densely packed arrangement, they may serve as acoustic sources when energized with radiation force, etc. Another example is use as a smart drug-delivery vehicle. By attaching Streptavidin-bound iron oxide particles to the biotinylated lipid of the PDMS shell, radio waves that heat only the iron oxide particles can potentially disrupt outer shell, releasing the drug. With stable PDMS bubbles composed of functionalized lipid within PDMS devices, it is possible to perform biodetection of antibodies, proteins, DNA/RNA, and other targets of interest. These compressible spherical PDMS microbubbles can be densely or loosely packed on-chip at specific locations depending on channel geometries. Incorporating phospholipid components within the PDMS shell enables a wide variety of targeting moieties to be incorporated (avidin-biotin, antibodies, DNA, magnetic beads, etc.) allowing for selective targeting for use as biosensors. High surface to volume ratios of the PDMS microbubbles improves detection speed. Reagents and analytes can be washed out, allowing the reuse of chips. PDMS-lipid microbubbles can be highly size-stable for more than six months of monitoring. Cell attachment can occur on a <15 μm PDMS-lipid microbubble, which dissolves quickly within 5-10 sec.

The foregoing descriptions of specific embodiments of the present invention have been presented for purposes of illustration and description. They are not intended to be exhaustive or to limit the invention to the precise forms disclosed, and many modifications and variations are possible in light of the above teaching. The embodiments were chosen and described in order to best explain the principles of the invention and its practical application, to thereby enable others skilled in the art to best utilize the invention and various embodiments with various modifications as are suited to the particular use contemplated. It is intended that the scope of the invention be defined by the claims appended hereto and their equivalents.

What is claimed is:

1. A microfluidic drug delivery system for producing multiple layer microbubbles comprising:
a first inlet for receiving a gas and directing said gas into a central stream;
a second inlet for receiving a first liquid containing a drug substance and flow focusing said first liquid around said gas;
a third inlet for receiving a second liquid and flow focusing said second liquid around said first liquid; and
a fourth inlet for receiving a third liquid and flow focusing said third liquid around said second liquid.

2. The microfluidic drug delivery system of claim 1 wherein said third liquid comprises a stabilizer.

3. The microfluidic drug delivery system of claim 1 wherein said third liquid comprises a surfactant.

4. The microfluidic drug delivery system of claim 1 further comprising:
a fifth inlet for delivering particles around said third liquid.

5. The microfluidic drug delivery system of claim 4 wherein said particles comprises a substance containing a metal.

6. A multiple layer microbubble drug delivery vehicle comprising:
a gas core;
a first liquid layer containing a drug, said first liquid layer surrounding said gas core;
a second liquid layer surrounding said first liquid layer for stabilizing said first liquid layer; and
a plurality of particles outside said second liquid layer.

7. The multiple layer microbubble drug delivery vehicle of claim 6 wherein said particles comprises magnetic particles.

8. The multiple layer microbubble drug delivery vehicle of claim 6 wherein said particles comprises iron oxide particles.

9. The multiple layer microbubble drug delivery vehicle of claim 6 wherein said particles comprises superparamagnetic iron oxide particles.

10. The multiple layer microbubble drug delivery vehicle of claim 6 wherein said particles comprises a substance containing a metal.

11. The multiple layer microbubble drug delivery vehicle of claim 6 wherein said particles comprises streptavidin coated particles.

12. The multiple layer microbubble drug delivery vehicle of claim 6 wherein said particles comprises avidin coated particles.

13. The multiple layer microbubble drug delivery vehicle of claim 6 wherein said particles comprises streptavidin coated iron oxide particles.

14. The multiple layer microbubble drug delivery vehicle of claim 6 further comprising:
a third liquid layer surrounding said second liquid layer for stabilizing said second liquid layer.

15. A method for treatment of disease in humans comprising:
introducing multiple layer drug delivery vehicles into blood;
concentrating said drug delivery vehicles at a target site; and
applying radio frequency energy to such target site to focus said drug delivery vehicles.

16. The method of claim 15 further comprising:
applying radio frequency energy to such target site to disrupt said drug delivery vehicles.

17. The method of claim 15 further comprising:
applying radio frequency energy to such target site to melt said drug delivery vehicles.
